(19) United States
(12) Patent Application Publication
(10) Pub. No.: US 2026/0253438 A1
(43) Pub. Date: Aug. 27, 2026

(54) SYSTEM

(71) Applicant: SoftBank Group Corp., Tokyo (JP)

(72) Inventor: Yo OE, Tokyo (JP)

(73) Assignee: SoftBank Group Corp., Tokyo (JP)

(21) Appl. No.: 19/539,054

(22) Filed: Feb. 13, 2026

(30) Foreign Application Priority Data
Feb. 21, 2025 (JP) 2025-026965

(51) Int. Cl. G06V 30/19; G06F 16/2458; G06F 40/295; G06K 7/14; G06N 3/0464; G06N 3/08; G06V 10/82; G06V 40/16; G10L 25/63

(52) U.S. Cl. CPC G06V 30/191; G06F 16/2462; G06F 40/295; G06K 7/1443; G06N 3/0464; G06N 3/08; G06V 10/82; G06V 40/174; G10L 25/63

(57) ABSTRACT

A system according to an embodiment includes a recording unit, an identification unit, and a proposal unit. The recording unit records purchase information. The identification unit identifies a medicine corresponding to a symptom based on information recorded by the recording unit. The proposal unit proposes arrangement of an ambulance or a hospital visit based on the medicine identified by the identification unit.

### CROSS-REFERENCE TO RELATED APPLICATION

The present application claims priority to and incorporates by reference the entire contents of Japanese Patent Application No. 2025-026965 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there has been a problem that inventory management of medicines in a home is insufficient, and it is difficult to select an appropriate medicine corresponding to a symptom or to respond in an emergency.

### SUMMARY OF THE INVENTION

A system according to an embodiment includes a recording unit, an identification unit, and a proposal unit. The recording unit records purchase information. The identification unit identifies a medicine corresponding to a symptom based on information recorded by the recording unit. The proposal unit proposes arrangement of an ambulance or a hospital visit based on the medicine identified by the identification unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

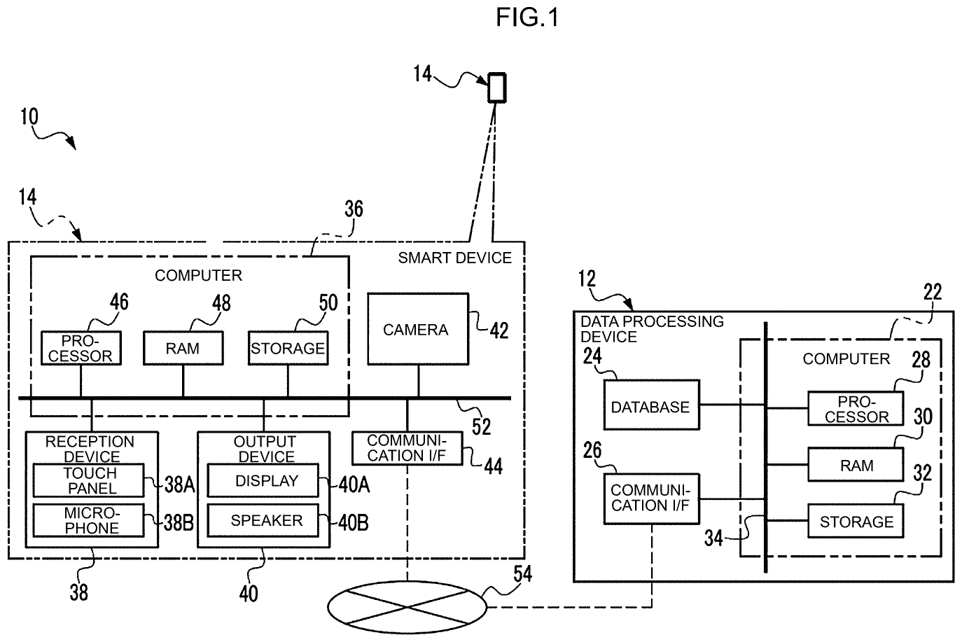

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

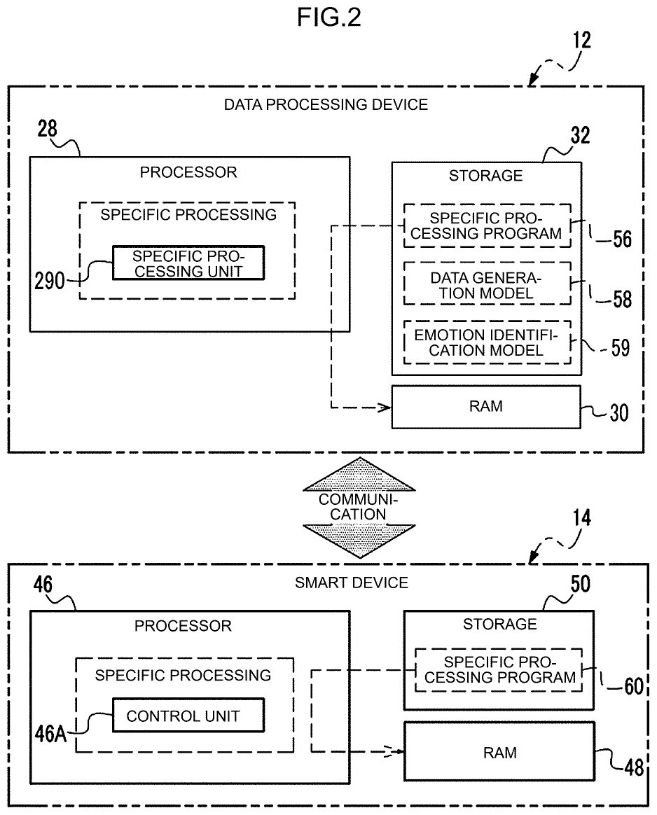

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The medicine management system according to the embodiment of the present invention is a system that manages purchase information of over-the-counter medicines and prescription medicines using a smartphone and proposes appropriate medication according to symptoms. This medicine management system records a purchase date and a stock status of a medicine by capturing an image of a receipt or reading a barcode when a user purchases an over-the-counter medicine. Next, for prescription medicines as well, the system performs character recognition by capturing an image of a prescription and stores storage information. Thereby, it is possible to centrally manage the stock status of medicines not only for an individual but also for the entire family. Furthermore, when the user inputs a symptom such as a cold, an AI automatically allocates a stored medicine and proposes appropriate medication. For example, when the user inputs “sore throat,” the AI identifies a medicine effective for a sore throat from among the stored medicines and proposes medication. At the same time, detailed information on the medicine is also reminded again. This allows the user to take an appropriate medicine quickly. Also, depending on the medical condition, the AI can recommend arrangement of an ambulance or a hospital visit. For example, when the user inputs “chest pain,” the AI determines severity of the symptom and proposes arrangement of an ambulance as necessary. In this way, the user's health management is supported. With this system, stock management of medicines becomes easy, and appropriate medication according to symptoms becomes possible. In addition, by centrally managing the stock status of medicines for the entire family, duplicate purchases or shortages of medicines can be prevented. Furthermore, since the AI recommends arrangement of an ambulance or a hospital visit according to symptoms, a prompt response becomes possible, and the user's health can be protected. Thereby, the medicine management system can support the user's health management and provide a prompt and appropriate response. Specifically, the present medicine management system adopts a client-server architecture composed of a mobile terminal possessed by the user and a server group on a cloud connected via a communication network such as the Internet, or an edge computing architecture. The present mobile terminal includes various input devices such as a high-resolution camera, a GPS sensor, and an acceleration sensor, and converts acquired physical data into digital signals for processing. An AI processing unit, which is the core of the present system, is implemented by a deep learning model such as a large-scale language model or an image recognition model operating on a parallel computing cluster using, for example, GPUs. The present AI processing unit includes an encoder that tokenizes natural language text (e.g., “I have a sore throat”) input from the user and converts it into a high-dimensional vector space (e.g., a 768-dimensional or 1024-dimensional embedding vector). The present encoder calculates a cosine similarity between an input symptom vector and a medicine vector indicating efficacy and effects of each medicine registered in a database in advance, and extracts a medicine whose similarity exceeds a predetermined threshold (e.g., 0.85) as a candidate. In addition, the present system executes optical character recognition (OCR) processing using a convolutional neural network (CNN) on a receipt image or a prescription image, identifies a character string region in the image, and converts it into text data. The text data obtained by the present conversion processing undergoes normalization processing and is stored in a stock management database as structured data (JSON format, etc.). Furthermore, the present system includes a classification model that performs sentiment analysis and urgency determination on the symptom text input by the user, and calculates an urgency score (a probability value in a range of 0 to 1) based on feature quantities extracted from the input text. When the present urgency score exceeds a predetermined danger threshold (e.g., 0.9), the present system generates an alert screen recommending immediate arrangement of an ambulance or a visit to a nearby emergency hospital, and performs control to display it on a display of the user terminal with the highest priority. Thus, the present system not only records and reproduces information but also combines image recognition, natural language processing, vector calculation, and inference algorithms in a complex manner, thereby assisting human judgment and providing an effect of technically supporting prompt decision-making related to human life in an emergency.

The medicine management system according to the embodiment includes a recording unit, an identification unit, and a proposal unit. The recording unit records purchase information. The purchase information includes, for example, a purchase date and time, a product name, a quantity, and a price, but is not limited to this example. The recording unit records a purchase date and a stock status of a medicine by capturing an image of a receipt or reading a barcode, for example, when a user purchases an over-the-counter medicine. In addition, the recording unit can perform character recognition for a prescription medicine by capturing an image of a prescription and store storage information. The identification unit identifies a medicine corresponding to a symptom based on the information recorded by the recording unit. For example, when the user inputs a symptom such as a cold, the identification unit automatically allocates a stored medicine using AI and proposes appropriate medication. The identification unit can also identify an optimal medicine based on data related to past symptoms and effects of medicines. For example, when the user inputs “sore throat,” the identification unit identifies a medicine effective for a sore throat from among stored medicines and proposes medication. The proposal unit proposes arrangement of an ambulance or a hospital visit based on the medicine identified by the identification unit. For example, when the user inputs “chest pain,” the proposal unit determines severity of the symptom using AI and proposes arrangement of an ambulance as necessary. The proposal unit can also determine severity of the symptom and propose arrangement of an ambulance or a hospital visit as necessary. Thereby, the medicine management system according to the embodiment can support the user's health management and provide a prompt and appropriate response. Part or all of the above-described processing in the recording unit, the identification unit, and the proposal unit may be performed using, for example, AI, or may be performed without using AI. For example, the recording unit can input the purchase information to the AI, and the AI can analyze the purchase information and record it. The identification unit can input the symptom to the AI, and the AI can identify the medicine based on the symptom. The proposal unit can propose arrangement of an ambulance or a hospital visit by the AI based on the identified medicine. Specifically, the present recording unit functions as a data processing pipeline that receives unstructured data such as image data and barcode data as input, analyzes them, and generates structured database records (including purchase date and time, medicine ID, quantity, expiration date, etc.). The present recording unit performs noise removal and geometric correction as preprocessing on input image data (e.g., tensor data having a pixel array of 8 bits for each of RGB), and then identifies a region where a product name or a price is described using an object detection model. The present identification unit includes an inference engine that performs matching between a user's symptom description (text data) and medicine information in a stock database. The present inference engine converts the symptom text into a distributed representation vector and searches for a stock record having a medicine vector closest to the symptom vector using a neighbor search algorithm (e.g., approximate nearest neighbor search) in a vector space. The present proposal unit executes decision-making logic for determining an action plan (medication instruction, hospital visit recommendation, emergency request, etc.) to be displayed on a user interface based on the identified medicine information and severity of the symptom. The present proposal unit inputs the input symptom text and vital data (if any) to a classifier such as a multilayer perceptron, and selects an optimal proposal by conditional branch processing based on output class classification probabilities (probabilities of mild, moderate, and severe). For example, when the probability of the severe class is maximum and equal to or greater than a threshold, the present proposal unit generates an interface for calling an ambulance arrangement API. Each of these units is implemented as a software module executed on hardware resources including a CPU, a memory, and a storage, and the modules exchange data via an API or a shared memory, thereby organically cooperating as the entire system to realize high-speed and high-precision medicine management and health support.

The medicine management system includes an imaging unit configured to capture an image of a receipt. The imaging unit captures an image of a receipt. The receipt includes, for example, a paper receipt, an electronic receipt, described information, and the like, but is not limited to this example. The imaging unit records a purchase date and a stock status of a medicine by capturing an image of a receipt, for example, when a user purchases an over-the-counter medicine. The imaging unit can capture an image of a receipt using, for example, a camera of a smartphone and save it as image data. Further, the imaging unit can analyze the image data of the receipt and extract purchase information. For example, the imaging unit extracts information such as a purchase date and time, a product name, a quantity, and a price from the image data of the receipt, and transmits the information to the recording unit. Thereby, the imaging unit can record the purchase information by capturing an image of the receipt. Part or all of the above-described processing in the imaging unit may be performed using, for example, AI, or may be performed without using AI. For example, the imaging unit can input the image data of the receipt to the AI, and the AI can extract the purchase information from the image data. Specifically, the present imaging unit has a capture function of controlling an image sensor such as a CMOS image sensor to convert an optical image of a target object (receipt) into digital image data (e.g., bitmap data in JPEG or PNG format). The present imaging unit inputs the acquired image data (input tensor: height×width×3 channels) to a trained image recognition model such as a convolutional neural network (CNN). The present image recognition model performs edge detection and region segmentation in the image, identifies a contour of the receipt, and executes geometric transformation processing such as keystone correction and rotation correction. Furthermore, the present imaging unit applies an optical character recognition (OCR) engine to the corrected image to convert pixel patterns in the image into a sequence of character codes (UTF-8, etc.). The present OCR engine has a function of recognizing words and contexts from a sequence of characters using a recurrent neural network (RNN) or LSTM (Long Short-Term Memory) and correcting misrecognition. The present imaging unit applies regular expression matching or a keyword extraction algorithm (e.g., logic for extracting numerical values around keywords such as “total,” “yen,” and “date”) to the extracted text data to generate structured data such as a purchase date and time, a product name, a unit price, and a quantity. This structured data is delivered to the recording unit in a format such as a JSON object. Thus, the present imaging unit not only saves an image but also automatically extracts meaningful information from a receipt image, which is unstructured data, by combining advanced image processing and natural language processing, thereby significantly reducing the trouble of manual input by the user and bringing about a technical effect of reducing input errors.

The medicine management system includes a reading unit configured to read a barcode. The reading unit reads the barcode. The barcode includes, for example, a QR code, but is not limited to this example. The reading unit records a purchase date and a stock status of a medicine by reading the barcode, for example, when a user purchases an over-the-counter medicine. The reading unit can read the barcode using, for example, a camera of a smartphone and save it as barcode data. Further, the reading unit can analyze the barcode data and extract purchase information. For example, the reading unit extracts information such as a purchase date and time, a product name, a quantity, and a price from the barcode data, and transmits the information to the recording unit. Thereby, the reading unit can record the purchase information by reading the barcode. Part or all of the above-described processing in the reading unit may be performed using, for example, AI, or may be performed without using AI. For example, the reading unit can input the barcode data to the AI, and the AI can extract the purchase information from the barcode data. Specifically, the present reading unit receives continuous frame images acquired from the camera as input, and detects an existence region of a barcode (one-dimensional barcode or two-dimensional code) at high speed using an image processing algorithm. The present reading unit performs binarization processing on the detected region and executes decoding processing for converting a pattern of black and white into a digital bit string. At this time, the present reading unit applies an image enhancement filter for correcting distortion and uneven illumination of the image to improve reading accuracy. The decoded bit string is converted into a numerical string or a character string according to a standard such as a JAN code (Japanese Article Number) or a GS1 code. The present reading unit issues a search query to an external product database or an internal product master table using the obtained identification code (e.g., a 13-digit number) as a key, and acquires detailed information such as a product name, a manufacturer, a capacity, and a classification of the corresponding medicine. Furthermore, the present reading unit can identify respective positions and perform decoding processing in parallel even when a plurality of barcodes exist in an image, using a deep learning-based object detection model. The acquired detailed information is formatted as structured data together with the purchase date and time (acquired from a system clock) and transmitted to the recording unit. Thus, the present reading unit integrates image analysis technology and database matching technology to instantly and accurately acquire digital information from a physical product package, thereby realizing automation and high precision of stock management.

The medicine management system includes a recognition unit configured to perform character recognition. The recognition unit performs character recognition. The character recognition includes, for example, OCR technology, handwritten character recognition, and the like, but is not limited to this example. The recognition unit performs character recognition by capturing an image of a prescription, for example, when a user purchases a prescription medicine, and records storage information. The recognition unit can capture an image of a prescription using, for example, a camera of a smartphone and save it as image data. Further, the recognition unit can analyze the image data and extract character information. For example, the recognition unit extracts contents of the prescription as character information from the image data and transmits the information to the recording unit. Thereby, the recognition unit can record information on the prescription medicine by performing character recognition. Part or all of the above-described processing in the recognition unit may be performed using, for example, AI, or may be performed without using AI. For example, the recognition unit can input the image data of the prescription to the AI, and the AI can extract the character information from the image data. Specifically, the present recognition unit implements a deep learning model that performs two-stage processing of text detection and text recognition on a prescription image (input data: high-resolution image tensor). First, in a text detection phase, a CNN-based model (e.g., a model having U-Net or ResNet as a backbone) predicts a bounding box of a character region in the image and separates a character portion from background noise. Next, in a text recognition phase, an image of each cut-out character region is input to a CRNN (Convolutional Recurrent Neural Network) or Transformer-based model to convert image feature quantities into a character sequence. At this time, the present recognition unit corrects an inference result using a language model specialized for medical terms to improve recognition accuracy of technical terms such as “antibiotic” and “dosage and administration.” Furthermore, the present recognition unit uses a layout analysis algorithm for analyzing a layout structure peculiar to a prescription (arrangement of patient name, medicine name, administration method, etc.) in combination, and maps a group of recognized texts to semantic fields (key-value pairs). For example, structured data such as “medicine name: Amlodipine” and “single dose: 5 mg” is generated. The generated structured data is saved in the recording unit as confirmed data after passing through a confirmation/correction interface by the user. Thus, the present recognition unit combines an advanced AI model and domain-specific post-processing logic to digitize information with high accuracy even from a prescription image containing handwritten characters or blurred printing, thereby simultaneously achieving reduction of input load and improvement of data quality.

The identification unit can identify an appropriate medicine based on data related to past symptoms and effects of medicines. The identification unit identifies an appropriate medicine based on data related to past symptoms and effects of medicines. The data related to past symptoms and effects of medicines includes, for example, electronic medical record data, patient self-reported data, and the like, but is not limited to this example. For example, when a user inputs a symptom such as a cold, the identification unit identifies an optimal medicine based on past data. For example, when the user inputs “sore throat,” the identification unit identifies a medicine effective for a sore throat based on past data and proposes medication. Thereby, the identification unit can propose appropriate medication by identifying an optimal medicine based on past data. Part or all of the above-described processing in the identification unit may be performed using, for example, AI, or may be performed without using AI. For example, the identification unit can input the data related to past symptoms and effects of medicines to the AI, and the AI can analyze the data to identify an optimal medicine. Specifically, the present identification unit refers to a history database including a user's personal past symptom history, taken medicines, and subsequent symptom improvement degrees (feedback data), and scores a medicine expected to be most effective for a current symptom using a recommendation algorithm such as collaborative filtering or matrix factorization. The present identification unit converts an input current symptom (text or option) into a feature vector and calculates similarity with a feature vector of a past successful case (a case where the symptom improved). Furthermore, the present identification unit can perform personalized recommendation considering the user's constitution and seasonal factors using a time-series prediction model using a recurrent neural network (RNN) or Transformer. For example, the present identification unit executes an inference model that receives a user ID, a current symptom vector, seasonal information, and a past effective medicine vector as input and outputs a recommendation score (0.0 to 1.0) for each stored medicine. This model assigns a high weight to history data indicating that “Medicine A” was effective for “sore throat” in the past, and preferentially displays “Medicine A” at the top of a recommendation list this time as well. In addition, the present identification unit cooperates with a drug interaction database, confirms whether there is any contraindication for concomitant use with other medicines currently being taken by filtering processing, and then outputs a final identification result. Thus, the present identification unit integrates pattern recognition of history data by machine learning and rule-based safety confirmation, rather than simple keyword search, thereby realizing medicine identification that is optimized for each user and has high medical validity.

The proposal unit can determine severity of the symptom and propose arrangement of an ambulance or a hospital visit as necessary. The proposal unit determines severity of the symptom and proposes arrangement of an ambulance or a hospital visit as necessary. The severity of the symptom includes, for example, duration of the symptom, intensity of pain, risk to life, and the like, but is not limited to this example. For example, when a user inputs “chest pain,” the proposal unit determines severity of the symptom and proposes arrangement of an ambulance as necessary. For example, when the user inputs “mild headache,” the proposal unit can also propose a hospital visit. Thereby, the proposal unit determines severity of the symptom and proposes an appropriate response, enabling a prompt response. Part or all of the above-described processing in the proposal unit may be performed using, for example, AI, or may be performed without using AI. For example, the proposal unit can input the severity of the symptom to the AI, and the AI can determine the severity and propose arrangement of an ambulance or a hospital visit. Specifically, the present proposal unit receives input symptom data (text, pain level value, duration, etc.) as feature quantities, and predicts an urgency level of the symptom (e.g., Level 1 “observation,” Level 2 “general outpatient visit,” Level 3 “emergency outpatient visit,” Level 4 “ambulance request”) using a multi-class classification model (e.g., random forest, gradient boosting decision tree, or deep neural network). This classification model is supervised-learned using a vast amount of past medical datasets (pairs of symptoms and diagnosis results), and is adjusted to output a high risk score for specific keywords (“chest pain,” “dyspnea,” “consciousness disorder,” etc.) or combinations thereof. For example, when the user inputs text “pain like chest tightening,” a natural language processing module in the present proposal unit analyzes this and extracts feature quantities suggesting a serious disease such as myocardial infarction. Subsequently, when the classification model calculates that a probability of being urgency level 4 (equivalent to ambulance request) is high (e.g., 95% or more) based on these feature quantities, the present proposal unit immediately displays a UI component capable of making an emergency call with one tap together with a visual warning such as turning the entire screen red. In addition, the present proposal unit also has a function of searching for a list of nearest emergency hospitals and night clinics using GPS location information and proposing route guidance in cooperation with a map application. Thus, the present proposal unit combines objective risk assessment by AI and location information service, thereby presenting an optimal action guideline based on medical evidence in real time without depending on the user's subjective judgment, and providing technical support for protecting the user's life and health.

The recording unit can estimate an emotion of a user and adjust a recording timing of the purchase information based on the estimated emotion of the user. The recording unit estimates an emotion of a user and adjusts a recording timing of the purchase information based on the estimated emotion of the user. The emotion of the user includes, for example, stress, relaxation, being in a hurry, and the like, but is not limited to this example. For example, when the user feels stress, the recording unit automatically records the purchase information to minimize user operations. For example, when the user is relaxed, the recording unit provides an option to manually record the purchase information so that the user can record it at his/her own pace. For example, when the user is in a hurry, the recording unit provides a simplified interface to record the purchase information quickly. Thereby, the recording unit can reduce the operation burden on the user by adjusting the recording timing of the purchase information according to the user's emotion. The estimation of the emotion is realized using an emotion estimation function using, for example, an emotion engine or generative AI. The generative AI is, for example, text generative AI (e.g., LLM) or multimodal generative AI, but is not limited to this example. Part or all of the above-described processing in the recording unit may be performed using, for example, AI, or may be performed without using AI. For example, the recording unit can input user's emotion data to the AI, and the AI can estimate the emotion and adjust the recording timing. Specifically, the present recording unit receives multimodal data including user's input text, voice data, facial expression images, or terminal operation logs (tap speed, scroll speed, etc.) as input, and outputs a user's current emotional state (e.g., impatience, calmness, fatigue) as a probability distribution using an emotion recognition model. The present emotion recognition model is composed of, for example, a multimodal encoder based on Transformer architecture, and integrates feature quantities of different modalities to classify emotion categories. The present recording unit dynamically changes UI/UX control logic based on the output emotional state. For example, when a probability of an “impatience” class is high, the present recording unit omits a confirmation dialog and switches to an “immediate recording mode” in which data is immediately saved by background processing. On the other hand, when a probability of a “calmness” class is high, a “detailed recording mode” is selected in which detailed input items are displayed and confirmation by the user is prompted. Also, when using generative AI (LLM), the present recording unit inputs a dialogue history with the user as a prompt, and the LLM infers a user's psychological state from the context and generates a proposal text such as “You seem to be in a hurry, so would you like to enter the details later?” and presents it to the user. Thus, the present recording unit senses an internal state called user's emotion and adaptively changes behavior (recording flow) of the system accordingly, thereby improving usability and realizing data input with less psychological load.

The recording unit can analyze a past purchase history of a user when recording the purchase information, and select an appropriate recording method. The recording unit analyzes a past purchase history of a user when recording the purchase information, and selects an appropriate recording method. The past purchase history includes, for example, a purchase date and time, a product name, a quantity, a price, and the like, but is not limited to this example. For example, the recording unit automatically displays medicines frequently purchased by the user in the past as candidates to simplify recording. For example, the recording unit analyzes purchase frequency of a specific medicine from the user's past purchase history and optimizes the recording method. For example, the recording unit adjusts a recording timing of the purchase information based on the user's past purchase history and proposes an optimal recording method. Thereby, the recording unit can select an optimal recording method by analyzing the user's past purchase history. Part or all of the above-described processing in the recording unit may be performed using, for example, AI, or may be performed without using AI. For example, the recording unit can input past purchase history data to the AI, and the AI can analyze the data to select an optimal recording method. Specifically, the present recording unit learns periodicity and tendency of purchase patterns from the user's purchase history data using a time-series data analysis algorithm (e.g., ARIMA model or LSTM network). The present recording unit receives current date and time and seasonal information as input, and predicts a list of medicines likely to be purchased next and their probabilities. When there is a medicine with a high predicted probability, the present recording unit sets the medicine as a default value in an input form or displays a “usual medicine” button, thereby enabling recording with one tap. In addition, the present recording unit identifies a combination of medicines (basket analysis) that tend to be purchased at the same time using association analysis (Apriori algorithm, etc.), and automatically presents the other medicine as a candidate when one medicine is input. Furthermore, the present recording unit analyzes whether the user tends to leave detailed memos in the past or tends to record only minimal information, and has a function of automatically switching a layout of an input screen (detailed mode or simple mode). Thus, the present recording unit utilizes big data called past action logs to predict the user's next action and optimizes the interface in advance, thereby providing technical effects of improving efficiency of input operation and preventing recording omission.

The recording unit can perform filtering based on a current health condition or living situation of a user when recording the purchase information. The recording unit performs filtering based on a current health condition or living situation of a user when recording the purchase information. The current health condition or living situation includes, for example, health checkup results, lifestyle habits, stress levels, and the like, but is not limited to this example. For example, when the user is in poor health, the recording unit preferentially records purchase information of important medicines. For example, the recording unit filters and records purchase information of necessary medicines according to the user's living situation. For example, the recording unit adjusts a recording method of the purchase information based on the user's health condition or living situation to perform optimal recording. Thereby, the recording unit can preferentially record necessary information according to the user's health condition or living situation. Part or all of the above-described processing in the recording unit may be performed using, for example, AI, or may be performed without using AI. For example, the recording unit can input user's health condition or living situation data to the AI, and the AI can analyze the data to perform filtering. Specifically, the present recording unit includes a risk assessment model that receives vital data (heart rate, blood pressure, number of steps, etc.) acquired from a wearable device or a healthcare app and a health profile including interview data as input, and evaluates a user's risk level. The present recording unit identifies items highly relevant to the user's current risk profile (e.g., antihypertensive drugs or low-salt foods for a user with hypertension risk) from among a large number of purchased items read from a receipt or a barcode, and executes filtering logic to preferentially record these in a database. For example, a machine learning model such as a decision tree or random forest is used to calculate a “recording importance score” for each item, and only items whose score is equal to or greater than a threshold are subject to automatic recording, while others are subject to confirmation by the user. In addition, the present recording unit also has a function of setting a warning flag and calling attention simultaneously with recording when a product containing a contraindicated component is detected based on the user's living situation (e.g., during pregnancy, during lactation, having allergies). Thus, the present recording unit intelligently selects information by incorporating not only static rules but also dynamically changing user's health condition as context, thereby efficiently accumulating only information truly valuable to the user and realizing maintenance of database quality and reduction of user's cognitive load.

The recording unit can estimate an emotion of a user and determine a priority of information to be recorded based on the estimated emotion of the user. The recording unit estimates an emotion of a user and determines a priority of information to be recorded based on the estimated emotion of the user. The priority of information to be recorded includes, for example, importance, urgency, frequency, and the like, but is not limited to this example. For example, when the user feels stress, the recording unit preferentially records important information and postpones other information. For example, when the user is relaxed, the recording unit records all information equally. For example, when the user is in a hurry, the recording unit quickly records only the most important information. Thereby, the recording unit can preferentially record important information by determining the priority of information to be recorded according to the user's emotion. The estimation of the emotion is realized using an emotion estimation function using, for example, an emotion engine or generative AI. The generative AI is, for example, text generative AI (e.g., LLM) or multimodal generative AI, but is not limited to this example. Part or all of the above-described processing in the recording unit may be performed using, for example, AI, or may be performed without using AI. For example, the recording unit can input user's emotion data to the AI, and the AI can estimate the emotion and determine the priority of information. Specifically, the present recording unit has an emotion recognition neural network that receives user's biological reactions (voice tone, typing speed, touch pressure, etc.) and facial expression analysis results as input vectors and classifies an emotional state (e.g., high arousal/unpleasant=stress, low arousal/pleasant=relaxation). The present recording unit executes a scheduling algorithm that manipulates a priority queue of data recording tasks based on the estimated emotional state. For example, when it is determined that the user is in a “high stress” state, the present algorithm assigns the highest priority to a recording task of essential items (medicine name, dosage) and lowers the priority of recording tasks of optional items (purchase store, memo, price) or temporarily suspends them. This concentrates system resources and user's attention resources on the most important tasks. Conversely, in the case of a “relaxation” state, the present algorithm assigns equal priority to all items and prompts recording of a complete dataset. Also, when using generative AI, the present recording unit generates a simplified confirmation screen together with an empathetic message such as “I will save only the minimum information for now” according to the user's emotion. Thus, the present recording unit reflects the user's psychological context in the task management of the system, thereby realizing optimal data collection according to the situation without impairing user experience (UX).

The recording unit can preferentially record highly relevant information in consideration of geographical location information of a user when recording the purchase information. The recording unit preferentially records highly relevant information in consideration of geographical location information of a user when recording the purchase information. The geographical location information includes, for example, GPS data, location information services, and the like, but is not limited to this example. For example, the recording unit preferentially records information on medicines purchased by the user in a specific area. For example, the recording unit preferentially records information on highly relevant medicines based on the user's geographical location information. For example, when the user is traveling, the recording unit preferentially records information on medicines purchased at a travel destination. Thereby, the recording unit can preferentially record highly relevant information based on the user's geographical location information. Part or all of the above-described processing in the recording unit may be performed using, for example, AI, or may be performed without using AI. For example, the recording unit can input geographical location information data to the AI, and the AI can analyze the data to preferentially record highly relevant information. Specifically, the present recording unit receives latitude and longitude information acquired from GPS, Wi-Fi access points, or beacons and semantic information of a place (POI: Point of Interest, e.g., pharmacy, hospital, home, travel destination) as input, and performs context-aware recording control. The present recording unit collates with a location information database, and activates a normal recording flow for regular prescription medicines when the current location is a “family pharmacy,” while applying logic to prioritize recording of over-the-counter medicines corresponding to acute symptoms (motion sickness, abdominal pain, etc.) when the current location is a “drugstore at a travel destination.” Furthermore, the present recording unit refers to region-specific epidemic disease data (open data, etc.), and when a medicine related to a disease prevalent in that region is purchased, records that information with an importance flag. For example, when an antipyretic is purchased in an influenza epidemic area, dynamic flow generation is performed to prompt body temperature recording as well. In addition, using an AI model (e.g., a Bayesian network that has learned correlation between place and behavior), the user's intention (e.g., emergency purchase or stock replenishment) is inferred from the location information, and granularity of information to be recorded is automatically adjusted. Thus, the present recording unit utilizes spatial context information to deeply understand the situation in which the user is placed, and provides a technical effect of automating recording and management of information optimal for that place.

The recording unit can analyze social media activity of a user and record relevant information when recording the purchase information. The recording unit analyzes social media activity of a user and records relevant information when recording the purchase information. The social media activity includes, for example, posted contents, the number of likes, the number of followers, and the like, but is not limited to this example. For example, the recording unit automatically records information on medicines shared by the user on social media. For example, the recording unit extracts and records information on relevant medicines from the user's social media activity. For example, the recording unit records information on relevant medicines based on symptoms mentioned by the user on social media. Thereby, the recording unit can record relevant information based on the user's social media activity. Part or all of the above-described processing in the recording unit may be performed using, for example, AI, or may be performed without using AI. For example, the recording unit can input social media activity data to the AI, and the AI can analyze the data to record relevant information. Specifically, the present recording unit acquires timeline and post data of a social media account (SNS) linked based on user's permission, and performs text mining using natural language processing (NLP) technology. The present recording unit extracts symptom keywords such as “headache” and “slight cold” and medicine names such as “aspirin” and “stomach medicine” from posted text by named entity recognition (NER) using a language model such as BERT. The present recording unit associates the extracted keywords with purchase information, adds context information such as “painkiller purchased on the day when headache was complained on SNS,” and records it in the database. In addition, the present recording unit analyzes a history of “likes” given by the user to posts related to specific health foods or supplements, and constructs a user's health interest level (interest graph). Based on this interest level, when a related product is purchased, an additional field for recording its effect or impression is automatically generated. Furthermore, the present recording unit can detect a medicine package from an image posted on SNS using object detection technology and use it as supplementary data for purchase records. Thus, the present recording unit incorporates external social data, integrates it with internal purchase data, and analyzes it, thereby automatically generating a rich health record in line with the user's life context and enhancing the value of data.

The identification unit can estimate an emotion of a user and adjust a method for identifying the medicine based on the estimated emotion of the user. The identification unit estimates an emotion of a user and adjusts a method for identifying the medicine based on the estimated emotion of the user. The method for identifying the medicine includes, for example, severity of symptoms, past effects, user's emotion, and the like, but is not limited to this example. For example, when the user feels stress, the identification unit identifies a medicine with a simple interface. For example, when the user is relaxed, the identification unit identifies a medicine while providing detailed information. For example, when the user is in a hurry, the identification unit provides a simplified interface for quickly identifying a medicine. Thereby, the identification unit can identify an appropriate medicine by adjusting the method for identifying the medicine according to the user's emotion. The estimation of the emotion is realized using an emotion estimation function using, for example, an emotion engine or generative AI. The generative AI is, for example, text generative AI (e.g., LLM) or multimodal generative AI, but is not limited to this example. Part or all of the above-described processing in the identification unit may be performed using, for example, AI, or may be performed without using AI. For example, the identification unit can input user's emotion data to the AI, and the AI can estimate the emotion and adjust the method for identifying the medicine. Specifically, the present identification unit includes an adaptive interface control unit that receives an emotion vector (e.g., probability distribution of [impatience, anxiety, anger, calmness]) estimated from user's input operation and biological information as input, and dynamically changes parameters of a medicine search algorithm and presentation logic of search results. When it is determined that the user feels “impatience” or “pain,” the present identification unit operates in an “emergency mode” in which filtering conditions of the search algorithm are tightened and only medicines with high immediate effect (short Tmax: time to reach maximum blood concentration) or medicines with proven track record in the past are displayed at the top. At this time, detailed explanations such as side effect information are collapsed, and only the most important information such as dosage and administration interval is displayed in a large size. On the other hand, when the user is “calm,” the present identification unit operates in a “detailed consideration mode” in which a wide range of options including alternatives such as generic drugs and herbal medicines are presented, and merits and demerits of each can be compared and considered. Also, utilizing generative AI (LLM), the identification unit identifies a medicine while eliciting the user's latent needs by performing narrowing down in a dialogue format tailored to the user's emotional state (e.g., asking “It's painful, isn't it? Would you prefer something that doesn't make you sleepy?”). Thus, the present identification unit treats the user's emotion as an important variable in the search process, thereby realizing advanced decision-making support that is close to the user, beyond simple information search.

The identification unit can refer to data related to past symptoms and effects of medicines to identify an appropriate medicine when identifying the medicine. The identification unit refers to data related to past symptoms and effects of medicines to identify an appropriate medicine when identifying the medicine. The data related to past symptoms and effects of medicines includes, for example, electronic medical record data, patient self-reported data, and the like, but is not limited to this example. For example, the identification unit analyzes the user's past symptoms and effects of medicines to identify an optimal medicine. For example, the identification unit identifies a medicine most effective for a specific symptom based on past data. For example, the identification unit refers to data related to the user's past symptoms and effects of medicines to improve accuracy of identifying the medicine. Thereby, the identification unit can identify an optimal medicine by referring to past data. Part or all of the above-described processing in the identification unit may be performed using, for example, AI, or may be performed without using AI. For example, the identification unit can input data related to past symptoms and effects of medicines to the AI, and the AI can analyze the data to identify an optimal medicine. Specifically, the present identification unit accesses a knowledge graph or a relational database accumulating a user's personal health history (PHR: Personal Health Record) and anonymized treatment data of a similar user group. The present identification unit executes a case-based reasoning (CBR) algorithm that searches for a case with high similarity from past cases using a current symptom vector as a query. Furthermore, the present identification unit calculates a fitness score of each candidate medicine using a personalized recommendation model (e.g., matrix factorization or neural collaborative filtering) that has learned the user's past reactions to each medicine (labels such as “effective,” “side effect,” “not effective”). For example, if there is a record that a side effect of stomach pain occurred with “ibuprofen” in the past, the present model significantly lowers the score of that medicine and instead raises the score of a medicine with less burden on the stomach such as “acetaminophen.” In addition, the present identification unit can predict seasonal symptoms (hay fever, etc.) by time-series data analysis, and identify and propose a preventive medicine before symptoms appear. Thus, the present identification unit utilizes a vast amount of past data as a knowledge source and performs scientific and personalized medicine identification based on individual constitution and past experience, thereby maximizing therapeutic effects and minimizing side effect risks.

The identification unit can improve accuracy of identification based on a current health condition or living situation of a user when identifying the medicine. The identification unit improves accuracy of identification based on a current health condition or living situation of a user when identifying the medicine. The current health condition or living situation includes, for example, health checkup results, lifestyle habits, stress levels, and the like, but is not limited to this example. For example, the identification unit identifies an optimal medicine in consideration of the user's current health condition. For example, the identification unit improves accuracy of identification based on the user's living situation. For example, the identification unit analyzes the user's health condition or living situation to improve accuracy of identifying the medicine. Thereby, the identification unit can improve accuracy of identifying the medicine according to the user's health condition or living situation. Part or all of the above-described processing in the identification unit may be performed using, for example, AI, or may be performed without using AI. For example, the identification unit can input user's health condition or living situation data to the AI, and the AI can analyze the data to improve accuracy of identification. Specifically, the present identification unit includes a context-aware inference model that receives a multidimensional feature vector integrating real-time vital data (heart rate, blood pressure, body temperature, sleep time, etc.) and static health attributes (age, sex, medical history, allergy information) as input. The present inference model is composed of, for example, a gradient boosting decision tree (GBDT) or a deep neural network, and predicts an influence of each medicine in the current physical condition. For example, when the user's heart rate is higher than usual (tachycardia tendency), the present identification unit excludes cold medicines containing caffeine that may burden the heart, and preferentially identifies non-caffeine medicines. Also, when the user is in a living situation context such as “driving” or “working” (estimated from GPS or calendar information), the priority of medicines containing components that cause drowsiness (antihistamines, etc.) is lowered, and herbal medicines that are less likely to cause drowsiness are ranked higher. Furthermore, when there is test value data of renal function or liver function, the present identification unit can incorporate logic to select a medicine with less burden on organs in consideration of metabolic pathways. Thus, the present identification unit comprehensively considers the physiological and social situation in which the user is placed, thereby identifying a medicine that is not only effective for symptoms but also “optimal and safe” for the current life and body with high accuracy.

The identification unit can estimate an emotion of a user and determine a priority of medicines to be identified based on the estimated emotion of the user. The identification unit estimates an emotion of a user and determines a priority of medicines to be identified based on the estimated emotion of the user. The priority of medicines includes, for example, importance, urgency, expectation of effect, and the like, but is not limited to this example. For example, when the user feels stress, the identification unit preferentially identifies important medicines. For example, when the user is relaxed, the identification unit identifies all medicines equally. For example, when the user is in a hurry, the identification unit quickly identifies only the most important medicines. Thereby, the identification unit can preferentially identify important medicines by determining the priority of medicines to be identified according to the user's emotion. The estimation of the emotion is realized using an emotion estimation function using, for example, an emotion engine or generative AI. The generative AI is, for example, text generative AI (e.g., LLM) or multimodal generative AI, but is not limited to this example. Part or all of the above-described processing in the identification unit may be performed using, for example, AI, or may be performed without using AI. For example, the identification unit can input user's emotion data to the AI, and the AI can estimate the emotion and determine the priority of medicines. Specifically, the present identification unit implements a learning to rank algorithm that uses an emotion score (e.g., anxiety degree 0.8, urgency degree 0.9) output from an emotion recognition module as a weighting coefficient. This algorithm re-ranks a candidate medicine list by performing weighting according to the user's emotional state as well as medical recommendation degree. For example, when the user's “urgency degree” is high, a positive bias is applied to the score of a medicine having immediate effect (short Tmax: time to reach maximum blood concentration) or a strong analgesic effect, and it is placed at the top of the list. Conversely, when the user feels “anxiety,” the weighting is adjusted to prioritize a medicine with few side effects and mild efficacy or a medicine with a sense of security of a famous brand. Also, when using generative AI (LLM), the present identification unit inputs a prompt including the user's emotional context (e.g., “The user is very anxious. Please list medicines that give a sense of security preferentially”) to the LLM, and presents medicines in the order inferred by the LLM. Thus, the present identification unit optimizes the presentation order of medicines so as to satisfy not only pharmacological effects but also user's psychological needs (want to be relieved, want to stop pain immediately, etc.), thereby improving user satisfaction and medication adherence.

The identification unit can identify an appropriate medicine in consideration of geographical location information of a user when identifying the medicine. The identification unit identifies an appropriate medicine in consideration of geographical location information of a user when identifying the medicine. The geographical location information includes, for example, GPS data, location information services, and the like, but is not limited to this example. For example, when the user is in a specific area, the identification unit preferentially identifies medicines available in that area. For example, the identification unit identifies an optimal medicine based on the user's geographical location information. For example, when the user is traveling, the identification unit identifies medicines available at a travel destination. Thereby, the identification unit can identify an optimal medicine based on the user's geographical location information. Part or all of the above-described processing in the identification unit may be performed using, for example, AI, or may be performed without using AI. For example, the identification unit can input geographical location information data to the AI, and the AI can analyze the data to identify an optimal medicine. Specifically, the present identification unit executes a stock search API that cooperates with a stock database of nearby pharmacies or drugstores in real time or quasi-real time based on the user's current location (latitude and longitude). The present identification unit lists medically appropriate medicine candidates for symptoms, then collates whether those medicines are “in stock” at stores within a radius of several kilometers of the current location, and performs filtering processing to preferentially identify and display medicines in stock. Furthermore, the present identification unit refers to a cross-border medicine database considering pharmaceutical regulations and sales names that differ depending on the country or region. For example, when the user is traveling abroad, the present identification unit identifies a local over-the-counter medicine (counterpart) containing the same components as the Japanese medicine usually taken, and proposes it together with the product name in the local language and a package image. In addition, using an AI model, the present identification unit infers whether the user is on the way home or on a business trip from the movement history, and makes an advanced location-based proposal such as prioritizing stock at a pharmacy near home if on the way home, or prioritizing stock near the current location if on a business trip. Thus, the present identification unit considers both medical appropriateness and physical availability, thereby realizing effective medicine identification that is not pie in the sky.

The identification unit can analyze social media activity of a user and identify a relevant medicine when identifying the medicine. The identification unit analyzes the social media activity of the user and identifies the relevant medicine when identifying the medicine. The social media activity includes, for example, posted content, the number of likes, the number of followers, and the like, but is not limited to such examples. The identification unit identifies a relevant medicine based on, for example, a symptom shared by the user on social media. The identification unit identifies an optimal medicine for a specific symptom from the social media activity of the user, for example. The identification unit identifies a relevant medicine based on, for example, a medicine mentioned by the user on social media. Thereby, the identification unit can identify a relevant medicine based on the social media activity of the user. Part or all of the above-described processing in the identification unit may be performed using, for example, AI, or may be performed without using AI. For example, the identification unit can input social media activity data into the AI, and the AI can analyze the data to identify a relevant medicine. Specifically, the present identification unit includes a social listening engine configured to analyze trend data on social media and a posting log of the user themselves. This engine uses Natural Language Processing (NLP) to detect topics related to currently prevalent infectious diseases (influenza, norovirus, etc.) or pollen dispersal situations, and generates a “disease trend map” for each region. The present identification unit refers to this trend map, and when posts regarding a specific symptom are rapidly increasing in a residential area of the user, raises a recommendation score of a medicine corresponding to the symptom. For example, during a period when posts saying “my eyes are itchy” are increasing, an anti-allergic eye drop is preferentially identified. In addition, the present identification unit also has a function of identifying a medicine recommended by an influencer or a friend trusted by the user (a medicine for which “Like” or retweet has been done) and adding it to a recommendation reason as social proof. Furthermore, a chronic tendency such as “having a headache” is extracted from past posts of the user themselves and utilized for profiling. In this way, the present identification unit incorporates social collective intelligence and individual social activity into medicine identification logic, thereby enabling medicine proposal that is timely and matches interests of the user.

The proposal unit can estimate an emotion of a user and adjust a presentation method of a proposal based on the estimated emotion of the user. The proposal unit estimates the emotion of the user and adjusts the presentation method of the proposal based on the estimated emotion of the user. The presentation method of the proposal includes, for example, wording, timing of the proposal, a format of the proposal, and the like, but is not limited to such examples. For example, when the user feels stressed, the proposal unit makes a simple and easy-to-understand proposal. For example, when the user is relaxed, the proposal unit makes a proposal including detailed information. For example, when the user is in a hurry, the proposal unit makes a quick and concise proposal. Thereby, the proposal unit can make an appropriate proposal by adjusting the presentation method of the proposal in accordance with the emotion of the user. The estimation of the emotion is realized using an emotion estimation function using, for example, an emotion engine or generative AI. The generative AI is, for example, text generative AI (e.g., LLM) or multimodal generative AI, but is not limited to such examples. Part or all of the above-described processing in the proposal unit may be performed using, for example, AI, or may be performed without using AI. For example, the proposal unit can input emotion data of the user into the AI, and the AI can estimate the emotion and adjust the presentation method of the proposal. Specifically, the present proposal unit takes a proposal content (core message) and an emotion label of the user (e.g., anxiety, anger, sadness) as inputs, and executes style transfer processing for converting a tone and manner (tone, literary style) of a text to be presented using a Natural Language Generation (NLG) model or an LLM. When the user feels “anxiety”, the present proposal unit generates an empathetic and receptive expression (e.g., “It must be painful. Let's see how it goes with this medicine”) to provide a sense of security. On the other hand, when the user is “in a hurry”, the present proposal unit generates a directive expression that omits greetings and modifiers and shows only a conclusion in bullet points (e.g., “Recommended medicine: Tablet A. Dosage: 1 tablet after meal”). In addition, regarding a visual presentation method (UI design), a font size, a color scheme, and a button arrangement are dynamically changed in accordance with the emotion. For example, when it is estimated that an elderly person feels anxiety, a screen with high visibility in which characters are enlarged and contrast is strengthened is generated. In this way, the present proposal unit optimizes not only the “content” of information but also “how to convey” it to a psychological state of the user, thereby enhancing receptivity of information and promoting smooth behavior change.

The proposal unit can determine severity of the symptom and propose arrangement of an ambulance or a hospital visit as necessary when making a proposal. The proposal unit determines the severity of the symptom and proposes arrangement of an ambulance or a hospital visit as necessary when making a proposal. The severity of the symptom includes, for example, duration of the symptom, intensity of pain, risk to life, and the like, but is not limited to such examples. For example, when the symptom of the user is severe, the proposal unit proposes arrangement of an ambulance. For example, when the symptom of the user is mild, the proposal unit proposes a hospital visit. The proposal unit determines, for example, the severity of the symptom of the user and proposes an optimal response. Thereby, the proposal unit determines the severity of the symptom and proposes an appropriate response, thereby enabling a prompt response. Part or all of the above-described processing in the proposal unit may be performed using, for example, AI, or may be performed without using AI. For example, the proposal unit can input severity data of the symptom into the AI, and the AI can analyze the data to propose arrangement of an ambulance or a hospital visit. Specifically, the present proposal unit implements a hybrid of a rule-based engine based on a medical triage protocol (e.g., JTAS or MANCHESTER triage system) and a risk prediction model based on deep learning. The present proposal unit calculates an “urgency score” and a “severity score” from the input symptom, vital signs, and medical history, and evaluates these on a matrix to determine a response action. For example, when there is chest pain accompanied by cold sweat, since suspicion of myocardial infarction is high, the risk prediction model outputs a class of “ambulance immediately” with a probability of 99%. Based on this output, the present proposal unit bypasses a normal medicine proposal screen, displays a button “Call an ambulance?” on a full screen, and prepares an API for making a 119 call with GPS information added in the background. In addition, in the case of a moderate symptom (e.g., high fever but consciousness is clear), the present proposal unit searches for a nearby medical institution capable of medical examination at present, and presents it together with waiting time information and reservation availability. In this way, by incorporating medical urgency determination logic into the system, the present proposal unit immediately provides an optimal medical access route according to a situation even to a user who does not have specialized knowledge, contributing to improvement of a survival rate and appropriate use of medical resources.

The proposal unit can refer to a past health history of a user and select an appropriate proposal method when making a proposal. The proposal unit refers to the past health history of the user and selects the appropriate proposal method when making a proposal. The past health history includes, for example, a medical history, a prescription history, a medical record, and the like, but is not limited to such examples. The proposal unit selects an optimal proposal method based on, for example, the past health history of the user. The proposal unit makes an optimal proposal for a specific symptom from, for example, the past health history of the user. The proposal unit refers to, for example, the past health history of the user and improves accuracy of the proposal. Thereby, the proposal unit can select an optimal proposal method by referring to the past health history of the user. Part or all of the above-described processing in the proposal unit may be performed using, for example, AI, or may be performed without using AI. For example, the proposal unit can input past health history data into the AI, and the AI can analyze the data to select an optimal proposal method. Specifically, the present proposal unit includes a time-series data analysis module configured to analyze a Long-term Health Record (LHR) of the user. This module uses a Recurrent Neural Network (RNN) or LSTM to learn a sequence of a past medical history, a treatment course, and a drug reaction, and models a “Health Trajectory” unique to the user. The present proposal unit uses this model to identify whether the current symptom is temporary, an exacerbation of a chronic disease, or a sign of a new disease. For example, when a user with a history of asthma complains of “cough”, the present proposal unit preferentially proposes use of a bronchodilator or a visit to a respiratory medicine department instead of a mere cold medicine. In addition, for a user whose adherence (medication compliance rate) was low in the past, a support function such as “Do you want to set a forgotten-to-take prevention alarm?” is added at the time of proposal. Furthermore, a safety mechanism that collates an allergy history and a side effect history with a database and automatically excludes a risky proposal is also provided. In this way, the present proposal unit considers not only the current symptom as a point but also the past history as a line, thereby realizing a safe and effective proposal deeply adapted to a constitution and a medical history of the user.

The proposal unit can estimate an emotion of a user and determine a priority of proposals based on the estimated emotion of the user. The proposal unit estimates the emotion of the user and determines the priority of proposals based on the estimated emotion of the user. The priority of proposals includes, for example, importance, urgency, expected degree of effect, and the like, but is not limited to such examples. For example, when the user feels stressed, the proposal unit preferentially makes an important proposal. For example, when the user is relaxed, the proposal unit makes all proposals equally. For example, when the user is in a hurry, the proposal unit quickly makes only the most important proposal. Thereby, the proposal unit can preferentially make an important proposal by determining the priority of proposals in accordance with the emotion of the user. The estimation of the emotion is realized using an emotion estimation function using, for example, an emotion engine or generative AI. The generative AI is, for example, text generative AI (e.g., LLM) or multimodal generative AI, but is not limited to such examples. Part or all of the above-described processing in the proposal unit may be performed using, for example, AI, or may be performed without using AI. For example, the proposal unit can input emotion data of the user into the AI, and the AI can estimate the emotion and determine the priority of proposals. Specifically, the present proposal unit has a scoring engine configured to multiply a medical importance score and a weighting coefficient based on an emotion state of the user for a plurality of proposal candidates (e.g., medication, rest, hydration, hospital visit) to calculate a comprehensive score. When it is estimated that the user is in a “panic” state, this engine lowers a score of a proposal requiring complex judgment (e.g., comparative examination of a plurality of over-the-counter medicines) and significantly raises a score of a simple and clear action instruction (e.g., take a deep breath, call an emergency consultation counter). Thereby, cognitive load on the user is reduced and confusion is prevented. Conversely, when the user is in a state of being “calm” and “highly motivated to learn”, this engine raises a priority of a proposal for improvement of lifestyle habits or information on preventive medicine, and makes a proposal contributing to long-term health promotion. In addition, the present proposal unit also has a function of cooperating with a notification system to push-notify a suspended proposal with low importance (e.g., reminder for inventory replenishment) at a timing when the emotion state of the user is good. In this way, the present proposal unit maximizes effectiveness of the proposal by sorting information according to receptivity of the user.

The proposal unit can select an appropriate proposal method in consideration of geographical location information of a user when making a proposal. The proposal unit selects the appropriate proposal method in consideration of the geographical location information of the user when making a proposal. The geographical location information includes, for example, GPS data, a location information service, and the like, but is not limited to such examples. For example, when the user is in a specific region, the proposal unit proposes a medical institution available in the region. The proposal unit selects an optimal proposal method based on, for example, the geographical location information of the user. For example, when the user is traveling, the proposal unit proposes a medical institution available at a travel destination. Thereby, the proposal unit can select an optimal proposal method based on the geographical location information of the user. Part or all of the above-described processing in the proposal unit may be performed using, for example, AI, or may be performed without using AI. For example, the proposal unit can input geographical location information data into the AI, and the AI can analyze the data to select an optimal proposal method. Specifically, the present proposal unit takes GPS coordinates as an input and issues a spatial query to a map database and a medical institution database. The present proposal unit proposes an optimal consultation destination in a ranking format in consideration of a travel time from a current location to each medical institution, a means of transportation, and a current congestion situation (real-time waiting time data). For example, when the user develops a symptom in an “unfamiliar land (travel destination)” at “night”, the present proposal unit preferentially extracts an emergency hospital that is currently open for medical examination and satisfies conditions such as being capable of dealing with foreigners and accepting credit cards, and proposes it together with navigation to there. In addition, when proposing a drug store, the present proposal unit identifies and guides not only a nearby store but also a store having a stock of a necessary medicine. Furthermore, a function of advising an appropriate consultation method (necessity of a letter of introduction, etc.) in consideration of a difference in a medical system (insurance coverage, etc.) for each region can be incorporated. In this way, the present proposal unit integrates location information and external dynamic information, thereby specifically presenting a solution that is physically accessible to the user and optimal for the situation.

The proposal unit can analyze social media activity of a user and make a relevant proposal when making a proposal. The proposal unit analyzes the social media activity of the user and makes the relevant proposal when making a proposal. The social media activity includes, for example, posted content, the number of likes, the number of followers, and the like, but is not limited to such examples. The proposal unit makes a relevant proposal based on, for example, a symptom shared by the user on social media. The proposal unit makes an optimal proposal for a specific symptom from the social media activity of the user, for example. The proposal unit makes a relevant proposal based on, for example, a symptom mentioned by the user on social media. Thereby, the proposal unit can make a relevant proposal based on the social media activity of the user. Part or all of the above-described processing in the proposal unit may be performed using, for example, AI, or may be performed without using AI. For example, the proposal unit can input social media activity data into the AI, and the AI can analyze the data to make a relevant proposal. Specifically, the present proposal unit analyzes an activity log (posted text, shared article, participating community) on SNS of the user, and profiles health consciousness, lifestyle, and values of the user. For example, when the user belongs to a community related to “natural remedy” or “organic”, the present proposal unit proposes a coping method by herbal tea, Chinese herbal medicine, or lifestyle improvement (sleep, meal) in addition to a proposal of a chemical pharmaceutical product. In addition, when the user has posted on SNS that “I am too busy with work to go to a hospital”, the present proposal unit proposes use of an online medical examination application or a night medical examination clinic near an office. Furthermore, a coping method highly evaluated on SNS by other users having similar symptoms (e.g., “this stretch was effective”) is extracted and added to the proposal as word-of-mouth information, thereby enhancing conviction of the user. In this way, the present proposal unit reads a potential preference or constraint condition of the user from social data and makes a personalized proposal matching it, thereby improving user engagement.

The imaging unit can estimate an emotion of a user and adjust a timing of capturing an image based on the estimated emotion of the user. The imaging unit estimates the emotion of the user and adjusts the timing of capturing an image based on the estimated emotion of the user. The timing of capturing an image includes, for example, an emotion state of the user, an environmental condition, and the like, but is not limited to such examples. For example, when the user feels stressed, the imaging unit automatically performs imaging to minimize operation of the user. For example, when the user is relaxed, the imaging unit provides an option to perform imaging manually, allowing the user to perform imaging at their own pace. For example, when the user is in a hurry, the imaging unit provides a simplified interface to perform imaging quickly. Thereby, the imaging unit can reduce an operation burden on the user by adjusting the timing of capturing an image in accordance with the emotion of the user. The estimation of the emotion is realized using an emotion estimation function using, for example, an emotion engine or generative AI. The generative AI is, for example, text generative AI (e.g., LLM) or multimodal generative AI, but is not limited to such examples. Part or all of the above-described processing in the imaging unit may be performed using, for example, AI, or may be performed without using AI. For example, the imaging unit can input emotion data of the user into the AI, and the AI can estimate the emotion and adjust the timing of capturing an image. Specifically, the present imaging unit includes a gaze tracking/facial expression recognition module configured to analyze a facial expression or movement of a line of sight of the user from a camera preview image, and a motion analysis module configured to detect camera shake or intensity of movement from a gyro sensor of a terminal. When it is determined that the user is “irritated” (facial expression is grim, moving the terminal violently), the present imaging unit activates a “continuous shooting mode” or a “best shot automatic selection function”, and enhances an auto-capture function of automatically performing imaging at the moment when a target object (receipt or affected part) comes into a frame even if the user does not press a shutter button accurately. On the other hand, when the user is “relaxed” and trying to perform imaging carefully, the present imaging unit displays a grid line or a level and provides a guide function for adjusting composition. In addition, the generative AI is used to adjust a tone of voice guidance such as “a little more to the right” or “captured beautifully” in accordance with the emotion of the user to support an imaging experience. In this way, the present imaging unit dynamically optimizes control logic of a camera in accordance with a psychological and physical state of the user, thereby preventing failure of imaging and providing a stress-free input experience.

The imaging unit can refer to a past imaging history of a user and select an appropriate imaging method when capturing an image. The imaging unit refers to the past imaging history of the user and selects the appropriate imaging method when capturing an image. The past imaging history includes, for example, an imaging date and time, an imaging place, an imaging target, and the like, but is not limited to such examples. For example, the imaging unit automatically displays a method frequently used for imaging by the user in the past as a candidate to simplify imaging. The imaging unit analyzes a specific imaging method from, for example, the past imaging history of the user and proposes an optimal imaging method. The imaging unit adjusts an imaging timing based on, for example, the past imaging history of the user and selects an optimal imaging method. Thereby, the imaging unit can select an optimal imaging method by referring to the past imaging history of the user. Part or all of the above-described processing in the imaging unit may be performed using, for example, AI, or may be performed without using AI. For example, the imaging unit can input past imaging history data into the AI, and the AI can analyze the data to select an optimal imaging method. Specifically, the present imaging unit holds learning data in which metadata (exposure time, ISO sensitivity, white balance, presence/absence of flash use) of an image captured in the past is associated with a recognition success rate (OCR accuracy, etc.) of the image. The present imaging unit uses reinforcement learning or supervised learning to predict an imaging parameter setting with which recognition accuracy was highest in the past for a current ambient light or a type of a target object (receipt, prescription, affected part). For example, when there is a history that the user tends to capture an image of a receipt in a dark room and an OCR success rate was higher when a flash was turned on at that time, the present imaging unit automatically turns on the flash or applies a night mode under a similar illuminance environment. In addition, when the user has a habit of capturing an image from a specific angle, an initial parameter of keystone correction is adjusted in accordance with the angle to shorten a processing time. In this way, the present imaging unit learns an imaging habit or an environmental characteristic for each user and automatically optimizes a camera setting, thereby constantly acquiring high-quality image data and raising accuracy of subsequent recognition processing.

The imaging unit can estimate an emotion of a user and determine a priority of imaging based on the estimated emotion of the user. The imaging unit estimates the emotion of the user and determines the priority of imaging based on the estimated emotion of the user. The priority of imaging includes, for example, importance, urgency, necessity of imaging, and the like, but is not limited to such examples. For example, when the user feels stressed, the imaging unit preferentially performs important imaging. For example, when the user is relaxed, the imaging unit performs all imaging equally. For example, when the user is in a hurry, the imaging unit quickly performs only the most important imaging. Thereby, the imaging unit can preferentially perform important imaging by determining the priority of imaging in accordance with the emotion of the user. The estimation of the emotion is realized using an emotion estimation function using, for example, an emotion engine or generative AI. The generative AI is, for example, text generative AI (e.g., LLM) or multimodal generative AI, but is not limited to such examples. Part or all of the above-described processing in the imaging unit may be performed using, for example, AI, or may be performed without using AI. For example, the imaging unit can input emotion data of the user into the AI, and the AI can estimate the emotion and determine the priority of imaging. Specifically, the present imaging unit includes a workflow engine configured to dynamically configure an order of an imaging flow based on an emotion state of the user in a situation where a plurality of imaging targets (e.g., package of medicine, receipt, affected part, insurance card) exist. When the user feels “impatience” or “pain”, this engine presents imaging of a most important image directly connected to diagnosis or treatment (e.g., state of affected part, package of medicine taken just now) as a top priority task, and postpones imaging of a clerical image (e.g., insurance card, receipt) or makes it skippable. On the other hand, when the user is “calm”, this engine presents a full-course imaging flow for leaving a complete record including clerical work. In addition, prioritization is also performed for upload of imaging data and analysis processing. In an emergency, an instruction is issued to a backend scheduler to preferentially process analysis of an important image and return a result immediately. In this way, the present imaging unit changes weighting of a task in accordance with psychological allowance of the user, thereby ensuring usability in an emergency while reliably collecting necessary data.

The imaging unit can select an appropriate imaging method in consideration of geographical location information of a user when capturing an image. The imaging unit selects the appropriate imaging method in consideration of the geographical location information of the user when capturing an image. The geographical location information includes, for example, GPS data, a location information service, and the like, but is not limited to such examples. For example, when the user is in a specific region, the imaging unit proposes an imaging method optimal in the region. The imaging unit selects an optimal imaging method based on, for example, the geographical location information of the user. For example, when the user is traveling, the imaging unit proposes an imaging method optimal at a travel destination. Thereby, the imaging unit can select an optimal imaging method based on the geographical location information of the user. Part or all of the above-described processing in the imaging unit may be performed using, for example, AI, or may be performed without using AI. For example, the imaging unit can input geographical location information data into the AI, and the AI can analyze the data to select an optimal imaging method. Specifically, the present imaging unit includes environment estimation logic configured to estimate an environmental characteristic (indoor/outdoor, type of lighting, weather) of a current place from GPS information and date and time information. For example, when the location information indicates “in a hospital”, the present imaging unit automatically applies “manner mode imaging” in which a shutter sound is muted or minimized. In addition, when the location information is “outdoor” and “night”, the present imaging unit applies a setting that enhances camera shake correction and increases ISO sensitivity. Furthermore, in consideration of the fact that formats of receipts and prescriptions differ depending on the region, an appropriate image recognition template (setting of ROI: Region of Interest) is loaded based on the location information. For example, if the user is traveling abroad, switching to an OCR model and an imaging guide frame corresponding to the language and currency of that country is performed. In this way, the present imaging unit adapts behavior of a camera to a characteristic of a physical place, thereby reducing influence of environmental noise and enabling constant capture of an optimal image.

The reading unit can estimate an emotion of a user and adjust a timing of reading based on the estimated emotion of the user. The reading unit estimates the emotion of the user and adjusts the timing of reading based on the estimated emotion of the user. The timing of reading includes, for example, an emotion state of the user, an environmental condition, and the like, but is not limited to such examples. For example, when the user feels stressed, the reading unit automatically performs reading to minimize operation of the user. For example, when the user is relaxed, the reading unit provides an option to perform reading manually, allowing the user to perform reading at their own pace. For example, when the user is in a hurry, the reading unit provides a simplified interface to perform reading quickly. Thereby, the reading unit can reduce an operation burden on the user by adjusting the timing of reading in accordance with the emotion of the user. The estimation of the emotion is realized using an emotion estimation function using, for example, an emotion engine or generative AI. The generative AI is, for example, text generative AI (e.g., LLM) or multimodal generative AI, but is not limited to such examples. Part or all of the above-described processing in the reading unit may be performed using, for example, AI, or may be performed without using AI. For example, the reading unit can input emotion data of the user into the AI, and the AI can estimate the emotion and adjust the timing of reading. Specifically, the present reading unit monitors emotional arousal and valence of the user in real time from a camera image or sensor data. When it is determined that the user is in a state of high arousal and unpleasantness (irritation, impatience), the present reading unit maximizes a scan rate (FPS) of barcode detection and shifts to a “high-speed scan mode” in which reading is forcibly performed with a strong correction algorithm even if there is some blur or defocus. At this time, feedback (sound or vibration) upon successful reading is also made short and clear. On the other hand, when the user is relaxed, the present reading unit operates in a normal mode with reduced battery consumption, and performs interactive interaction such as proposing use of a “continuous scan mode” for continuously reading a plurality of product codes. In addition, the generative AI is used to display or voice-output specific advice such as “please move a little further away” or “please try in a bright place” in polite language that does not upset the emotion of the user who is having trouble reading. In this way, the present reading unit adjusts responsiveness and interactivity of the system in accordance with the emotion of the user, thereby providing a stress-free reading experience.

The reading unit can refer to a past reading history of a user and select an appropriate reading method when reading. The reading unit refers to the past reading history of the user and selects the appropriate reading method when reading. The past reading history includes, for example, a reading date and time, a reading place, a reading target, and the like, but is not limited to such examples. For example, the reading unit automatically displays a method frequently used for reading by the user in the past as a candidate to simplify reading. The reading unit analyzes a specific reading method from, for example, the past reading history of the user and proposes an optimal reading method. The reading unit adjusts a reading timing based on, for example, the past reading history of the user and selects an optimal reading method. Thereby, the reading unit can select an optimal reading method by referring to the past reading history of the user. Part or all of the above-described processing in the reading unit may be performed using, for example, AI, or may be performed without using AI. For example, the reading unit can input past reading history data into the AI, and the AI can analyze the data to select an optimal reading method. Specifically, the present reading unit accumulates a log of reading operations (distance, angle, lighting condition, type of barcode used) successfully performed by the user in the past as learning data. Based on this learning data, the present reading unit predicts a reading setting with the highest success probability in a current scene. For example, when the user frequently uses a unique QR code of a specific drug store, upon detecting that the user is in the store by GPS, the present reading unit preferentially activates a decoding algorithm optimized for the unique code instead of a standard JAN code reader. In addition, for a product for which there is a history that the user failed to read a barcode and switched to manual input in the past, a manual input form is displayed together from the beginning, or a more sensitive reading mode is automatically applied. In this way, the present reading unit feeds back past success and failure experiences to the system and performs personalized reading control tailored to a habit or an environment of each user, thereby aiming for efficiency of operation.

The reading unit can estimate an emotion of a user and determine a priority of reading based on the estimated emotion of the user. The reading unit estimates the emotion of the user and determines the priority of reading based on the estimated emotion of the user. The priority of reading includes, for example, importance, urgency, necessity of reading, and the like, but is not limited to such examples. For example, when the user feels stressed, the reading unit preferentially performs important reading. For example, when the user is relaxed, the reading unit performs all reading equally. For example, when the user is in a hurry, the reading unit quickly performs only the most important reading. Thereby, the reading unit can preferentially perform important reading by determining the priority of reading in accordance with the emotion of the user. The estimation of the emotion is realized using an emotion estimation function using, for example, an emotion engine or generative AI. The generative AI is, for example, text generative AI (e.g., LLM) or multimodal generative AI, but is not limited to such examples. Part or all of the above-described processing in the reading unit may be performed using, for example, AI, or may be performed without using AI. For example, the reading unit can input emotion data of the user into the AI, and the AI can estimate the emotion and determine the priority of reading. Specifically, the present reading unit implements object detection/selection logic that performs prioritization (triage) of processing targets based on an emotion state of the user when a plurality of reading targets (e.g., barcodes of a plurality of medicines, QR codes, character information) exist in an image. When the user is in an “impatience” state, this logic decodes an object in the center of a screen or an object with the largest size (presumed to be a main medicine) with top priority, and omits or delays processing of surrounding small codes or character information. Thereby, a response time is shortened. On the other hand, when the user is “calm”, this logic performs batch processing of comprehensively detecting all codes in the screen and listing and displaying them collectively. In addition, the generative AI is used to infer an intention of the user (whether “want to know details of this medicine” or “simply want to record”) from the emotion, and dynamically switch whether to prioritize detailed information acquisition processing or recording processing. In this way, the present reading unit allocates limited calculation resources and time of the user to a task that is most valuable in the psychological state at that time.

The reading unit can select an appropriate reading method in consideration of geographical location information of a user when reading. The reading unit selects the appropriate reading method in consideration of the geographical location information of the user when reading. The geographical location information includes, for example, GPS data, a location information service, and the like, but is not limited to such examples. For example, when the user is in a specific region, the reading unit proposes a reading method optimal in the region. The reading unit selects an optimal reading method based on, for example, the geographical location information of the user. For example, when the user is traveling, the reading unit proposes a reading method optimal at a travel destination. Thereby, the reading unit can select an optimal reading method based on the geographical location information of the user. Part or all of the above-described processing in the reading unit may be performed using, for example, AI, or may be performed without using AI. For example, the reading unit can input geographical location information data into the AI, and the AI can analyze the data to select an optimal reading method. Specifically, the present reading unit identifies a major barcode standard used in the country or region (e.g., UPC in North America, EAN in Europe, JAN in Japan) or a unique code system used in a specific drug store chain based on the location information, and automatically switches a setting of a decoding engine. For example, when the user is in a pharmacy overseas, the present reading unit changes a connection destination to a local drug code database and loads a parser corresponding to a local code system. In addition, a lighting environment (e.g., whether it is a dim warehouse-type store or a bright specialty store) is estimated from the location information, and exposure compensation or an autofocus mode of a camera is optimized in advance. Furthermore, a security function of referring to information on counterfeit medicines distributed in a specific region and verifying whether a read code is genuine can be enhanced in conjunction with the location information. In this way, the present reading unit dynamically reconfigures a reading algorithm and a reference database in accordance with a context of a place, thereby realizing global and robust reading performance.

The recognition unit can estimate an emotion of a user and adjust accuracy of character recognition based on the estimated emotion of the user. The recognition unit estimates the emotion of the user and adjusts the accuracy of character recognition based on the estimated emotion of the user. The accuracy of character recognition includes, for example, a setting of a recognition algorithm, an environmental condition, and the like, but is not limited to such examples. For example, when the user feels stressed, the recognition unit increases the accuracy of character recognition to reduce misrecognition. For example, when the user is relaxed, the recognition unit performs processing with normal character recognition accuracy. For example, when the user is in a hurry, the recognition unit adjusts the accuracy to perform character recognition quickly. Thereby, the recognition unit can reduce misrecognition by adjusting the accuracy of character recognition in accordance with the emotion of the user. The estimation of the emotion is realized using an emotion estimation function using, for example, an emotion engine or generative AI. The generative AI is, for example, text generative AI (e.g., LLM) or multimodal generative AI, but is not limited to such examples. Part or all of the above-described processing in the recognition unit may be performed using, for example, AI, or may be performed without using AI. For example, the recognition unit can input emotion data of the user into the AI, and the AI can estimate the emotion and adjust the accuracy of character recognition. Specifically, the present recognition unit has a parameter tuning function of dynamically controlling a processing mode (high accuracy/low speed mode vs low accuracy/high speed mode) of an OCR engine based on an emotion score of the user. When the user is “irritated” (tolerance for misrecognition is low), the present recognition unit selects a high-accuracy deep learning model with high calculation cost (e.g., large-scale Transformer-based model) and further thoroughly performs post-processing by dictionary matching or context analysis, thereby giving top priority to accuracy even if a processing time is slightly extended. Conversely, when the user is “in a hurry” but “emotion is stable”, the present recognition unit selects a lightweight model (e.g., MobileNet-based model) and performs high-speed processing emphasizing real-time performance. In addition, the generative AI is used to determine whether to honestly convey “this part could not be read” or to present a guess candidate and let the user choose, in accordance with the emotion of the user when there is uncertainty in a recognition result. In this way, the present recognition unit optimizes a trade-off between accuracy and speed in accordance with a psychological state of the user, thereby maximizing subjective satisfaction.

The recognition unit can refer to a past recognition history of a user and select an appropriate recognition method when performing recognition. The recognition unit refers to the past recognition history of the user and selects the appropriate recognition method when performing recognition. The past recognition history includes, for example, a recognition date and time, a recognition place, a recognition target, and the like, but is not limited to such examples. For example, the recognition unit automatically displays a character frequently recognized by the user in the past as a candidate to simplify recognition. The recognition unit analyzes a specific recognition method from, for example, the past recognition history of the user and proposes an optimal recognition method. The recognition unit adjusts a recognition timing based on, for example, the past recognition history of the user and selects an optimal recognition method. Thereby, the recognition unit can select an optimal recognition method by referring to the past recognition history of the user. Part or all of the above-described processing in the recognition unit may be performed using, for example, AI, or may be performed without using AI. For example, the recognition unit can input past recognition history data into the AI, and the AI can analyze the data to select an optimal recognition method. Specifically, the present recognition unit has a learning function of holding and updating a correction dictionary or a language model personalized for each user. The present recognition unit learns a recognition result corrected by the user in the past (e.g., history that “aspirin” was misrecognized as “asporin” and corrected by the user), and applies the correction pattern to automatically correct it at the time of next recognition. In addition, the present recognition unit learns a prescription layout of a medical institution frequently used by the user or a feature of a package font of an over-the-counter medicine frequently purchased, and preferentially applies a recognition template specialized for them. Furthermore, the present recognition unit can also learn a habit (handwriting feature) of handwritten characters of the user from the past history to improve recognition accuracy of handwritten input or a handwritten prescription. In this way, the present recognition unit constructs an adaptive recognition system that is optimized for the user as it is used, thereby improving long-term convenience.

The recognition unit can estimate an emotion of a user and determine a priority of recognition based on the estimated emotion of the user. The recognition unit estimates the emotion of the user and determines the priority of recognition based on the estimated emotion of the user. The priority of recognition includes, for example, importance, urgency, necessity of recognition, and the like, but is not limited to such examples. For example, when the user feels stressed, the recognition unit preferentially performs important recognition. For example, when the user is relaxed, the recognition unit performs all recognition equally. For example, when the user is in a hurry, the recognition unit quickly performs only the most important recognition. Thereby, the recognition unit can preferentially perform important recognition by determining the priority of recognition in accordance with the emotion of the user. The estimation of the emotion is realized using an emotion estimation function using, for example, an emotion engine or generative AI. The generative AI is, for example, text generative AI (e.g., LLM) or multimodal generative AI, but is not limited to such examples. Part or all of the above-described processing in the recognition unit may be performed using, for example, AI, or may be performed without using AI. For example, the recognition unit can input emotion data of the user into the AI, and the AI can estimate the emotion and determine the priority of recognition. Specifically, the present recognition unit performs semantic segmentation for assigning semantic importance to a plurality of text blocks (e.g., medicine name, dosage and administration, precautions, hospital name) included in an image. When the user feels “impatience” or “anxiety”, the present recognition unit concentrates CPU/GPU resources on recognition processing of information essential for taking medicine such as “medicine name” and “dosage and administration”, and presents these to the user fastest. On the other hand, incidental information such as “precautions” and “hospital address” is processed with delay in the background or recognized only when requested by the user. In addition, the generative AI is used to generate a summary that highlights a keyword related to a current emotion state of the user (e.g., “side effect” if anxious, “efficacy” if painful) from recognized text. In this way, the present recognition unit performs prioritization and presentation of information based on psychological needs of the user instead of listing information, thereby preventing cognitive overload.

The recognition unit can select an appropriate recognition method in consideration of geographical location information of a user when performing recognition. The recognition unit selects the appropriate recognition method in consideration of the geographical location information of the user when performing recognition. The geographical location information includes, for example, GPS data, a location information service, and the like, but is not limited to such examples. For example, when the user is in a specific region, the recognition unit proposes a recognition method optimal in the region. The recognition unit selects an optimal recognition method based on, for example, the geographical location information of the user. For example, when the user is traveling, the recognition unit proposes a recognition method optimal at a travel destination. Thereby, the recognition unit can select an optimal recognition method based on the geographical location information of the user. Part or all of the above-described processing in the recognition unit may be performed using, for example, AI, or may be performed without using AI. For example, the recognition unit can input geographical location information data into the AI, and the AI can analyze the data to select an optimal recognition method. Specifically, the present recognition unit automatically identifies a language of a recognition target (Japanese, English, Chinese, etc.), a currency symbol, and a date format based on GPS information, and switches a language model of an OCR engine. For example, when the user is in France, the present recognition unit loads a French medical term dictionary and supports character recognition including accent marks. In addition, accuracy of field extraction (identification of medicine name and dose) is improved by acquiring a standard format (layout) of a prescription different for each region from a database and performing appropriate template matching based on the location information. Furthermore, the present recognition unit optimizes a pre-processing filter for image recognition in consideration of a feature of a package design of a medicine distributed in the region. In this way, the present recognition unit utilizes location information as a context of a recognition process, thereby supporting global medicine management corresponding to a multilingual and multicultural environment.

The system according to the embodiment is not limited to the above-described examples, and various modifications are possible, for example, as follows. Specifically, the present system can be implemented not only as a monolithic application but also as a distributed system based on a microservices architecture, and each functional module (recording, identification, proposal, etc.) is deployed as an independent container and managed by an orchestration tool such as Kubernetes, whereby scalability and fault tolerance can be ensured. In addition, the present system can use not only a smartphone but also various IoT devices such as smart glasses, a smart watch, a home robot, and a smart speaker as a client, and can provide UI/UX tailored to device characteristics of each.

The medicine management system can analyze a user's medication history and detect drug interactions. For example, the recording unit collects data on medicines taken by the user in the past, and the identification unit determines whether a medicine proposed for a current symptom has a possibility of interacting with past medicines. If the risk of interaction is high, the proposal unit can propose an alternative medicine. Thereby, the user can take the medicine safely. Specifically, the present system includes a Drug-Drug Interaction (DDI) prediction engine constructed using a deep learning model such as a Graph Neural Network (GNN). This engine takes as input a graph structure having chemical structural formulas (SMILES notation, etc.) of medicines and target protein information as nodes, and solves the presence or absence of interaction between medicines and the type thereof (effect enhancement, effect attenuation, side effect manifestation) as a link prediction problem. The present identification unit calculates a risk score using this DDI prediction engine for all combinations of a list of medicines currently being taken by the user and a medicine that has become a proposal candidate this time. When the risk score exceeds a threshold, the present proposal unit displays a warning and searches for and proposes an alternative medicine with a low risk of interaction (a medicine with another ingredient having the same efficacy) from a knowledge graph. Thereby, a high-level safety check including unknown interactions becomes possible.

The medicine management system can acquire meal information of a user and adjust a timing of taking a medicine. For example, the recording unit cooperates with an application in which the user records meals to acquire meal contents. The identification unit determines whether there is a possibility of affecting absorption of the medicine based on the meal contents. The proposal unit can make a proposal to adjust the timing of taking the medicine in accordance with the meal contents. Thereby, the effects of the medicine can be maximized. Specifically, the present system includes a meal image recognition AI, and estimates a dish name and nutrients (lipids, carbohydrates, caffeine content, etc.) from a meal photo captured by the user. The present identification unit uses a Pharmacokinetics (PK) simulation model to predict an influence of estimated meal components on an absorption rate (bioavailability) and a metabolic rate of the medicine. For example, when a high-fat diet is detected, a proposal to finely adjust a dosage in consideration of increased absorption of a fat-soluble medicine, or a proposal to shift a taking time (e.g., wait 2 hours after a meal) for a medicine whose absorption is inhibited is made. In addition, an interaction between a specific food such as grapefruit and a medicine (CYP3A4 inhibition, etc.) is detected, and a specific alert is issued. In this way, optimization of drug efficacy is aimed at by considering chemical environmental factors called meals.

The medicine management system can acquire exercise information of a user and adjust a dosage of a medicine. For example, the recording unit cooperates with an application in which the user records exercise to acquire an amount of exercise. The identification unit determines whether there is a possibility of affecting metabolism of the medicine based on the amount of exercise. The proposal unit can make a proposal to adjust the dosage of the medicine in accordance with the amount of exercise. Thereby, the effects of the medicine can be optimized. Specifically, the present system analyzes activity amount data (number of steps, calories burned, heart rate transition, exercise intensity METs) acquired from a smartwatch or a fitness tracker as time-series data. The present identification unit evaluates an influence of blood flow increase and hypermetabolism due to exercise on pharmacokinetics of the medicine using a mathematical model. For example, when a user using insulin or a hypoglycemic agent performs vigorous exercise, it predicts that a risk of hypoglycemia increases, and proposes a complementary meal or a reduction in dosage. In addition, it detects dehydration due to sweating, and strongly encourages hydration when taking a medicine having a diuretic effect. In this way, safety management in line with lifestyle habits is realized by feeding back dynamic physical activity data to pharmacotherapy.

The medicine management system can acquire sleep information of a user and adjust a timing of taking a medicine. For example, the recording unit cooperates with an application in which the user records sleep to acquire sleep data. The identification unit determines whether there is a possibility of affecting effects of the medicine based on the sleep data. The proposal unit can make a proposal to adjust the timing of taking the medicine in accordance with the sleep data. Thereby, the effects of the medicine can be maximized. Specifically, the present system analyzes sleep quality data such as sleep stages (REM sleep, non-REM sleep), sleep time, and the number of awakenings acquired from a wearable device. The present identification unit learns a correlation between a timing of taking a medicine such as a sleeping pill or an anti-anxiety drug and sleep quality, and identifies a taking time (e.g., 30 minutes before bedtime, 1 hour before bedtime) at which the highest quality sleep is obtained for the user. In addition, when a tendency that the quality of sleep is deteriorating is detected, it proposes changing a taking time of a medicine that may cause insomnia as a side effect (such as steroids and some antidepressants) from evening to morning. In this way, a chronotherapeutic approach using sleep data contributes to improvement of QOL.

The medicine management system can acquire a stress level of a user and adjust a timing of taking a medicine. For example, the recording unit cooperates with an application in which the user records stress to acquire stress data. The identification unit determines whether there is a possibility of affecting effects of the medicine based on the stress data. The proposal unit can make a proposal to adjust the timing of taking the medicine in accordance with the stress data. Thereby, the effects of the medicine can be maximized. Specifically, the present system quantifies autonomic nerve balance and a stress level in real time using data from heart rate variability (HRV) analysis or an electrodermal activity (EDA) sensor. The present identification unit considers that gastric acid secretion increases and gastrointestinal motility is suppressed in a high stress state (sympathetic nerve dominance), and makes a proposal to change a timing of taking a stomach medicine from before a meal to after a meal, or adjusts a taking interval in consideration of a possibility that an effect of an analgesic is attenuated. In addition, for headaches or abdominal pain caused by stress, it also makes a proposal to use a relaxation method (breathing method, etc.) together with pharmacotherapy. In this way, care from both mind and body aspects is realized by considering a psychophysiological state.

The medicine management system can estimate an emotion of a user and adjust a timing of taking a medicine based on the estimated emotion. For example, the recording unit estimates an emotion of the user, and the identification unit determines whether there is a possibility of affecting effects of the medicine based on emotion data. The proposal unit can make a proposal to adjust the timing of taking the medicine in accordance with the emotion data. Thereby, the effects of the medicine can be maximized. Specifically, the present system applies nudge theory in behavioral economics and implements a reinforcement learning model that determines an optimal timing (intervention point) for inducing medication taking behavior according to an emotional state of the user. For example, when the user is “depressed”, since energy to take an active action of taking medicine is low, the system temporarily postpones a reminder saying “Let's take it when you feel a little better”, or sends a notification with a message that strengthens motivation saying “You will feel better if you take this”. Conversely, when the user is in a “positive” state, it praises taking medicine on time to encourage habituation. In this way, improvement of adherence is aimed at by timing control considering a context of emotion.

The medicine management system can estimate an emotion of a user and adjust a dosage of a medicine based on the estimated emotion. For example, the recording unit estimates an emotion of the user, and the identification unit determines whether there is a possibility of affecting effects of the medicine based on emotion data. The proposal unit can make a proposal to adjust the dosage of the medicine in accordance with the emotion data. Thereby, the effects of the medicine can be optimized. Specifically, the present system includes logic for calculating a recommended dosage based on a subjective distress level (emotion score) of the user within a range of “use as needed (take appropriately according to symptoms)” described in a doctor's prescription instruction or a package insert. For example, when there is an instruction of “1 to 2 tablets at a time” for an analgesic, if it is determined that a distress level estimated from a facial expression or voice of the user is high (severe pain, panic), “2 tablets” is recommended, and if the distress level is low (bearable), “1 tablet” is recommended. At this time, a history data of past dosages and effects is also referred to, and a safety limiter is provided so as not to result in overdose. In this way, subjective emotion data is utilized as an auxiliary index for objective dosage determination.

The medicine management system can estimate an emotion of a user and adjust selection of a medicine based on the estimated emotion. For example, the recording unit estimates an emotion of the user, and the identification unit selects an optimal medicine based on emotion data. The proposal unit can make a proposal to adjust the selection of the medicine in accordance with the emotion data. Thereby, an optimal medicine according to the emotion of the user can be selected. Specifically, the present system uses a preference learning model that learns preferences of the user, and performs matching between an emotional state and characteristics of a medicine (shape, taste, immediate effect, sustainability). For example, when a child or an elderly person with difficulty swallowing shows “anxiety” or “refusal”, the present system preferentially selects and proposes an easy-to-drink syrup or dry syrup, or a jelly-like preparation, instead of a large tablet. Also, when a user sensitive to taste shows “discomfort”, a coated tablet with less bitterness is selected. In this way, by performing medicine selection considering sensory evaluation at the time of taking in addition to pharmacological action, a hurdle for taking is lowered.

The medicine management system can estimate an emotion of a user and adjust a method of taking a medicine based on the estimated emotion. For example, the recording unit estimates an emotion of the user, and the identification unit selects an optimal method of taking based on emotion data. The proposal unit can make a proposal to adjust the method of taking the medicine in accordance with the emotion data. Thereby, an optimal method of taking according to the emotion of the user can be selected. Specifically, the present system dynamically generates content and instruction details for medication assistance according to an emotional state of the user. For example, when the user is “nervous”, since there is a possibility that throat muscles contract and it becomes difficult to swallow, a procedure for encouraging relaxation such as “First, take a deep breath, drink a sip of water, and then take the medicine” is presented. Also, when the user feels “troublesome” (boredom, lethargy), gamification elements are incorporated, and by displaying point grant or a cheering message after taking medicine, a device is made to change the act of taking medicine itself into a positive experience. In this way, the entire experience surrounding the act of taking medicine is designed.

The medicine management system can estimate an emotion of a user and adjust a medicine taking reminder based on the estimated emotion. For example, the recording unit estimates an emotion of the user, and the identification unit selects an optimal reminder timing based on emotion data. The proposal unit can make a proposal to adjust the medicine taking reminder in accordance with the emotion data. Thereby, an optimal reminder according to the emotion of the user can be set. Specifically, the present system implements a reinforcement learning agent (e.g., DQN or PPO algorithm) in which an emotional state of the user is a state space, a notification timing is an action space, and medication success is a reward. This agent learns that if a notification is sent when the user is “concentrating (flow state)”, it is ignored or causes discomfort and a reward (taking medicine) is not obtained, and comes to send a notification at a timing when concentration is interrupted and the user is “relaxed”. In addition, a pattern of a notification sound or vibration is also adjusted according to the emotion, and a notification method that does not cause stress is autonomously acquired. In this way, by the AI reading the atmosphere of the user, an effective reminder that does not get in the way is realized.

The flow of processing of Example of the Embodiment will be briefly described below. Specifically, a series of data processing processes in the present system is started with a user's input as a trigger, and each AI module on the cloud cooperates as pipeline processing, and is configured as a sequential and parallel flow in which a final output is returned to a user terminal.

Step 1: The recording unit records purchase information. The purchase information includes a purchase date and time, a product name, a quantity, a price, and the like. When the user purchases an over-the-counter medicine, the purchase date and a stock state of the medicine are recorded by capturing an image of a receipt or reading a barcode. Also, for a prescription drug, storage information can be stored by capturing an image of a prescription to perform character recognition.

Step 2: The identification unit identifies a medicine corresponding to a symptom based on the information recorded by the recording unit. When the user inputs a symptom such as a cold, the AI automatically allocates a stored medicine and proposes appropriate medication. It is also possible to identify an optimal medicine based on data related to past symptoms and effects of medicines. For example, when the user inputs “sore throat”, the identification unit identifies a medicine effective for a sore throat from among stored medicines and proposes taking the medicine.

Step 3: The proposal unit proposes arrangement of an ambulance or a hospital visit based on the medicine identified by the identification unit. When the user inputs “chest pain”, the AI determines severity of the symptom and proposes arrangement of an ambulance as necessary. It is also possible to determine severity of the symptom and propose arrangement of an ambulance or a hospital visit as necessary. Specifically, in Step 1, the system executes real-time object detection and OCR processing on an image stream acquired from a camera device, normalizes extracted text data, and commits it as a transaction of a relational database. In the subsequent Step 2, a natural language query (symptom) input from a user interface is vectorized, similarity search (k-NN, etc.) with medicine vectors in a stock database is performed, and an optimal medicine candidate list is generated through filtering processing based on a health profile of the user. In the final Step 3, a severity level of the input symptom is inferred by a classification model, condition branching logic operates according to the score, and a different action such as normal advice display, search/guidance of a medical institution, or calling of an emergency call API is executed, and the result is rendered on a display of the user terminal.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of a plurality of elements including the above-described recording unit, identification unit, proposal unit, imaging unit, reading unit, and recognition unit is implemented by, for example, at least one of a smart device 14 and a data processing device 12. For example, the recording unit is implemented by a control unit 46A of the smart device 14 and records purchase information. The identification unit is implemented by, for example, an identification processing unit 290 of the data processing device 12 and identifies a medicine corresponding to a symptom. The proposal unit is implemented by, for example, the identification processing unit 290 of the data processing device 12 and proposes arrangement of an ambulance or a hospital visit. The imaging unit captures an image of a receipt using a camera 42 of the smart device 14 and records purchase information. The reading unit reads a barcode using the camera 42 of the smart device 14 and records purchase information. The recognition unit captures an image of a prescription using the camera 42 of the smart device 14 and records character information. The correspondence relationship between each unit and the device or the control unit is not limited to the above-described example, and various modifications are possible.

### Second Embodiment

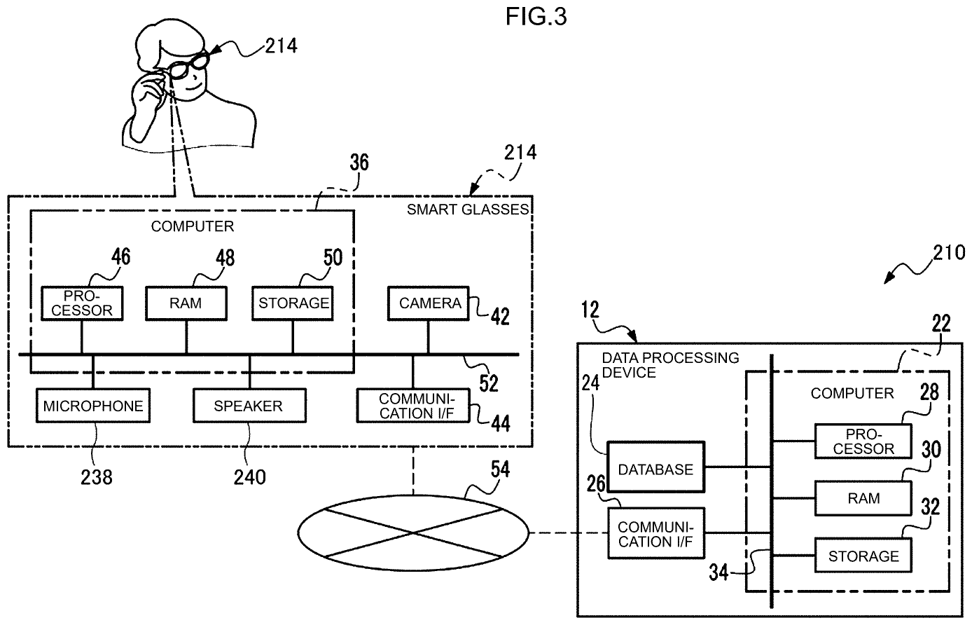

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

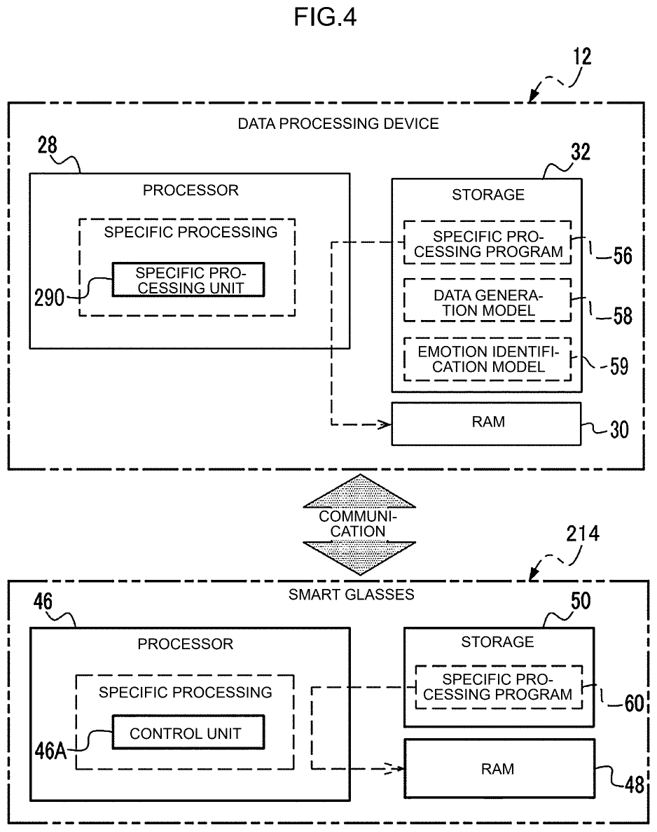

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of a plurality of elements including the above-described recording unit, identification unit, proposal unit, imaging unit, reading unit, and recognition unit is implemented by, for example, at least one of smart glasses 214 and the data processing device 12. For example, the recording unit is implemented by a control unit 46A of the smart glasses 214 and records purchase information. The identification unit is implemented by, for example, the identification processing unit 290 of the data processing device 12 and identifies a medicine corresponding to a symptom. The proposal unit is implemented by, for example, the identification processing unit 290 of the data processing device 12 and proposes arrangement of an ambulance or a hospital visit. The imaging unit captures an image of a receipt using a camera 42 of the smart glasses 214 and records purchase information. The reading unit reads a barcode using the camera 42 of the smart glasses 214 and records purchase information. The recognition unit captures an image of a prescription using the camera 42 of the smart glasses 214 and records character information. The correspondence relationship between each unit and the device or the control unit is not limited to the above-described example, and various modifications are possible.

### Third Embodiment

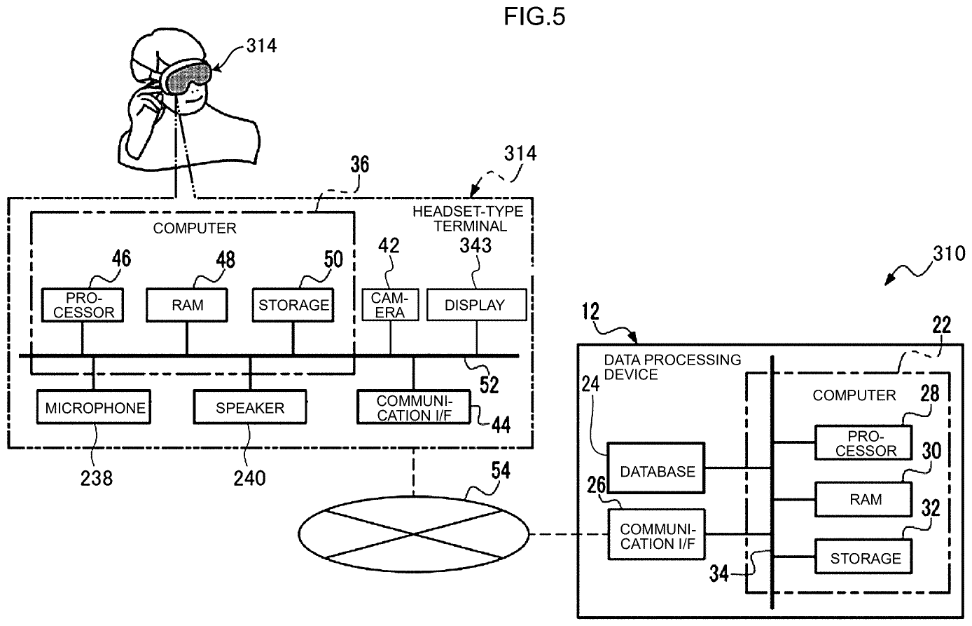

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

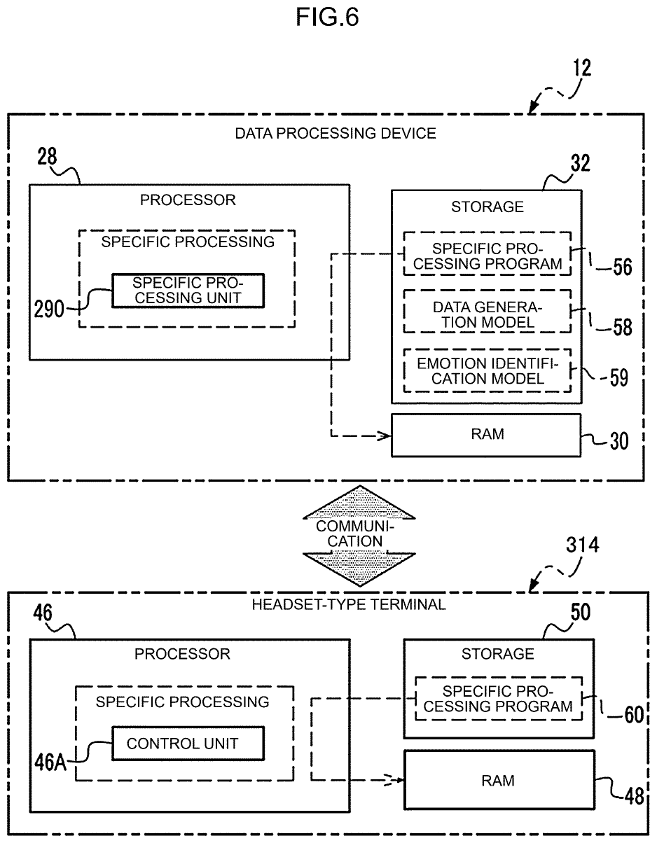

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of a plurality of elements including the above-described recording unit, identification unit, proposal unit, imaging unit, reading unit, and recognition unit is implemented by, for example, at least one of a headset-type terminal 314 and the data processing device 12. For example, the recording unit is implemented by a control unit 46A of the headset-type terminal 314 and records purchase information. The identification unit is implemented by, for example, the identification processing unit 290 of the data processing device 12 and identifies a medicine corresponding to a symptom. The proposal unit is implemented by, for example, the identification processing unit 290 of the data processing device 12 and proposes arrangement of an ambulance or a hospital visit. The imaging unit captures an image of a receipt using a camera 42 of the headset-type terminal 314 and records purchase information. The reading unit reads a barcode using the camera 42 of the headset-type terminal 314 and records purchase information. The recognition unit captures an image of a prescription using the camera 42 of the headset-type terminal 314 and records character information. The correspondence relationship between each unit and the device or the control unit is not limited to the above-described example, and various modifications are possible.

### Fourth Embodiment

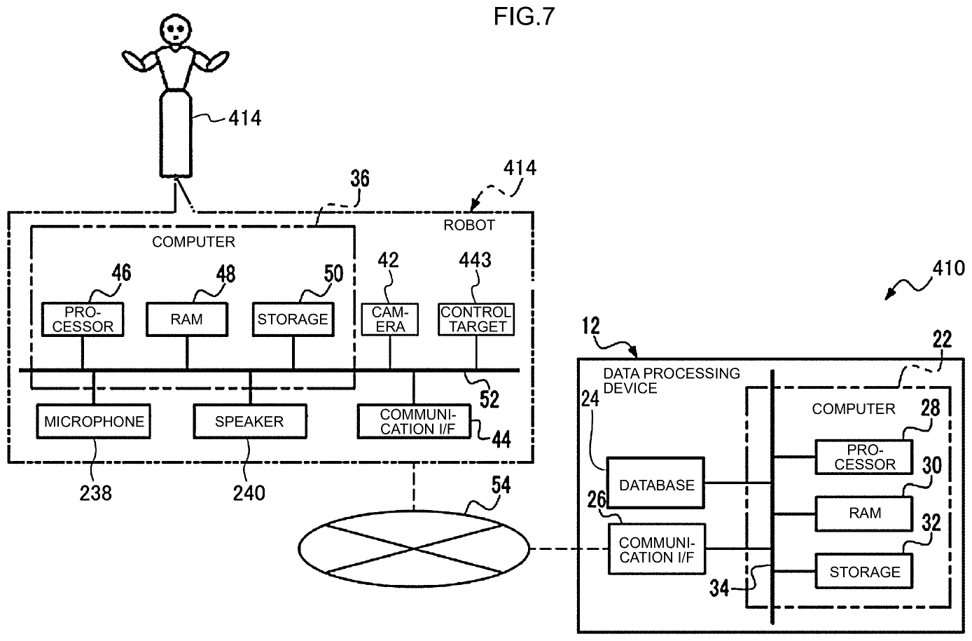

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

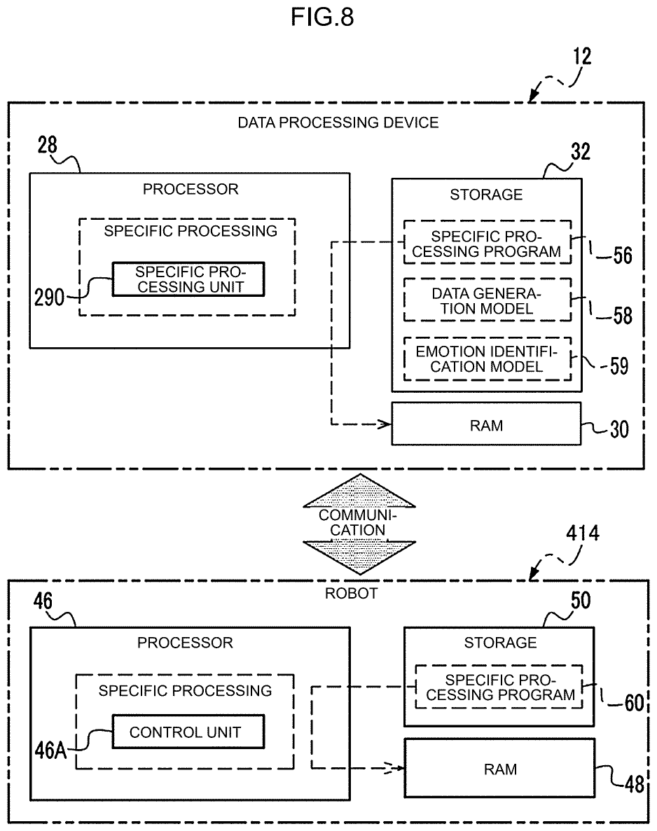

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of a plurality of elements including the above-described recording unit, identification unit, proposal unit, imaging unit, reading unit, and recognition unit is implemented by, for example, at least one of a robot 414 and the data processing device 12. For example, the recording unit is implemented by a control unit 46A of the robot 414 and records purchase information. The identification unit is implemented by, for example, the identification processing unit 290 of the data processing device 12 and identifies a medicine corresponding to a symptom. The proposal unit is implemented by, for example, the identification processing unit 290 of the data processing device 12 and proposes arrangement of an ambulance or a hospital visit. The imaging unit captures an image of a receipt using a camera 42 of the robot 414 and records purchase information. The reading unit reads a barcode using the camera 42 of the robot 414 and records purchase information. The recognition unit captures an image of a prescription using the camera 42 of the robot 414 and records character information. The correspondence relationship between each unit and the device or the control unit is not limited to the above-described example, and various modifications are possible.

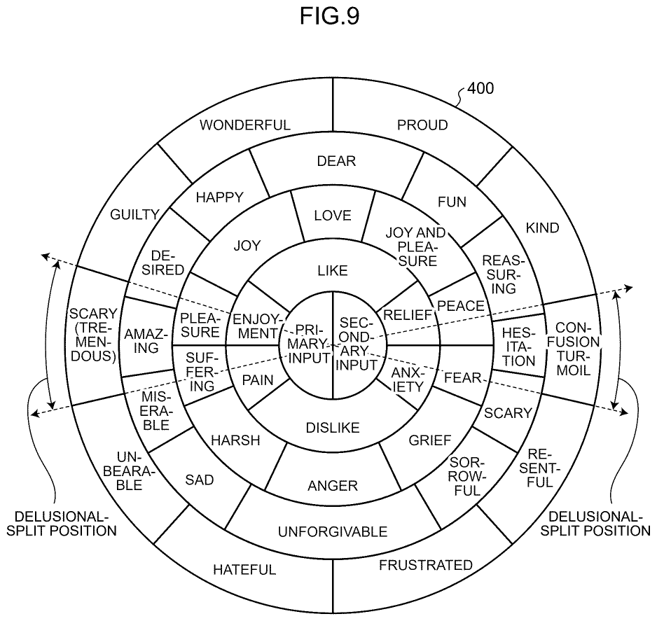

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

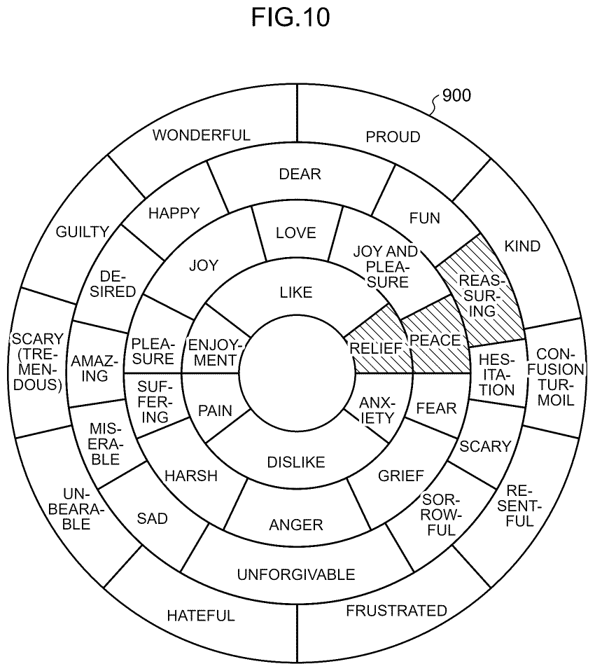

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

(Supplementary Note 1) A system comprising: a recording unit configured to record purchase information; an identification unit configured to identify a medicine corresponding to a symptom based on information recorded by the recording unit; and a proposal unit configured to propose arrangement of an ambulance or a hospital visit based on the medicine identified by the identification unit.

(Supplementary Note 2) The system according to Supplementary Note 1, further comprising an imaging unit configured to capture an image of a receipt.

(Supplementary Note 3) The system according to Supplementary Note 1, further comprising a reading unit configured to read a barcode.

(Supplementary Note 4) The system according to Supplementary Note 1, further comprising a recognition unit configured to perform character recognition.

(Supplementary Note 5) The system according to Supplementary Note 1, wherein the identification unit is configured to identify an appropriate medicine based on data related to past symptoms and effects of medicines.

(Supplementary Note 6) The system according to Supplementary Note 1, wherein the proposal unit is configured to determine severity of the symptom and propose arrangement of an ambulance or a hospital visit as necessary.

(Supplementary Note 7) The system according to Supplementary Note 1, wherein the recording unit is configured to estimate an emotion of a user and adjust a recording timing of the purchase information based on the estimated emotion of the user.

(Supplementary Note 8) The system according to Supplementary Note 1, wherein the recording unit is configured to analyze a past purchase history of a user when recording the purchase information, and select an appropriate recording method.

(Supplementary Note 9) The system according to Supplementary Note 1, wherein the recording unit is configured to perform filtering based on a current health condition or living situation of a user when recording the purchase information.

(Supplementary Note 10) The system according to Supplementary Note 1, wherein the recording unit is configured to estimate an emotion of a user and determine a priority of information to be recorded based on the estimated emotion of the user.

(Supplementary Note 11) The system according to Supplementary Note 1, wherein the recording unit is configured to preferentially record highly relevant information in consideration of geographical location information of a user when recording the purchase information.

(Supplementary Note 12) The system according to Supplementary Note 1, wherein the recording unit is configured to analyze social media activity of a user and record relevant information when recording the purchase information.

(Supplementary Note 13) The system according to Supplementary Note 1, wherein the identification unit is configured to estimate an emotion of a user and adjust a method for identifying the medicine based on the estimated emotion of the user.

(Supplementary Note 14) The system according to Supplementary Note 1, wherein the identification unit is configured to refer to data related to past symptoms and effects of medicines to identify an appropriate medicine when identifying the medicine.

(Supplementary Note 15) The system according to Supplementary Note 1, wherein the identification unit is configured to improve accuracy of identification based on a current health condition or living situation of a user when identifying the medicine.

(Supplementary Note 16) The system according to Supplementary Note 1, wherein the identification unit is configured to estimate an emotion of a user and determine a priority of medicines to be identified based on the estimated emotion of the user.

(Supplementary Note 17) The system according to Supplementary Note 1, wherein the identification unit is configured to identify an appropriate medicine in consideration of geographical location information of a user when identifying the medicine.

(Supplementary Note 18) The system according to Supplementary Note 1, wherein the identification unit is configured to analyze social media activity of a user and identify a relevant medicine when identifying the medicine.

(Supplementary Note 19) The system according to Supplementary Note 1, wherein the proposal unit is configured to estimate an emotion of a user and adjust a presentation method of a proposal based on the estimated emotion of the user.

(Supplementary Note 20) The system according to Supplementary Note 1, wherein the proposal unit is configured to determine severity of the symptom and propose arrangement of an ambulance or a hospital visit as necessary when making a proposal.

(Supplementary Note 21) The system according to Supplementary Note 1, wherein the proposal unit is configured to refer to a past health history of a user and select an appropriate proposal method when making a proposal.

(Supplementary Note 22) The system according to Supplementary Note 1, wherein the proposal unit is configured to estimate an emotion of a user and determine a priority of proposals based on the estimated emotion of the user.

(Supplementary Note 23) The system according to Supplementary Note 1, wherein the proposal unit is configured to select an appropriate proposal method in consideration of geographical location information of a user when making a proposal.

(Supplementary Note 24) The system according to Supplementary Note 1, wherein the proposal unit is configured to analyze social media activity of a user and make a relevant proposal when making a proposal.

(Supplementary Note 25) The system according to Supplementary Note 2, wherein the imaging unit is configured to estimate an emotion of a user and adjust a timing of capturing an image based on the estimated emotion of the user.

(Supplementary Note 26) The system according to Supplementary Note 2, wherein the imaging unit is configured to refer to a past imaging history of a user and select an appropriate imaging method when capturing an image.

(Supplementary Note 27) The system according to Supplementary Note 2, wherein the imaging unit is configured to estimate an emotion of a user and determine a priority of imaging based on the estimated emotion of the user.

(Supplementary Note 28) The system according to Supplementary Note 2, wherein the imaging unit is configured to select an appropriate imaging method in consideration of geographical location information of a user when capturing an image.

(Supplementary Note 29) The system according to Supplementary Note 3, wherein the reading unit is configured to estimate an emotion of a user and adjust a timing of reading based on the estimated emotion of the user.

(Supplementary Note 30) The system according to Supplementary Note 3, wherein the reading unit is configured to refer to a past reading history of a user and select an appropriate reading method when reading.

(Supplementary Note 31) The system according to Supplementary Note 3, wherein the reading unit is configured to estimate an emotion of a user and determine a priority of reading based on the estimated emotion of the user.

(Supplementary Note 32) The system according to Supplementary Note 3, wherein the reading unit is configured to select an appropriate reading method in consideration of geographical location information of a user when reading.

(Supplementary Note 33) The system according to Supplementary Note 4, wherein the recognition unit is configured to estimate an emotion of a user and adjust accuracy of character recognition based on the estimated emotion of the user.

(Supplementary Note 34) The system according to Supplementary Note 4, wherein the recognition unit is configured to refer to a past recognition history of a user and select an appropriate recognition method when performing recognition.

(Supplementary Note 35) The system according to Supplementary Note 4, wherein the recognition unit is configured to estimate an emotion of a user and determine a priority of recognition based on the estimated emotion of the user.

(Supplementary Note 36) The system according to Supplementary Note 4, wherein the recognition unit is configured to select an appropriate recognition method in consideration of geographical location information of a user when performing recognition.

What is claimed is:

1. A system comprising:
a communication interface configured to communicate with a client terminal via a packet-switched network;
a processor;
a random-access memory;
a memory storing a data generation model obtained by deep learning on a neural network, and an emotion identification model;
a database; and
circuitry configured to:
receive, from the client terminal via the communication interface, image data captured by a camera of the client terminal;
extract structured data from the image data by performing at least one of optical character recognition or code decoding using a convolutional neural network, and store the extracted structured data in the database;
receive, from the client terminal via the communication interface, query data comprising at least one of text data or voice data;
convert the query data into a feature vector, calculate a similarity between the feature vector and reference vectors stored in the database, and identify a record from the database based on the calculated similarity;
estimate an emotion of the user by applying the emotion identification model to sensor data received from the client terminal via the communication interface;
generate, using the data generation model, inference data comprising at least one of a recommendation text or an action proposal, based on the identified record and an urgency score calculated from the query data; and
transmit the inference data to the client terminal via the communication interface and the packet-switched network, the inference data causing the client terminal to present the inference data to the user.

2. The system according to claim 1, wherein the image data represents at least one of a receipt image, a barcode image, or a prescription image, and wherein extracting the structured data comprises identifying a character string region in the image data using an object detection model and converting the character string region into text data using the optical character recognition.

3. The system according to claim 1, wherein the code decoding comprises detecting a code region in the image data, performing binarization processing on the code region, and converting a pattern into a digital identification code, and wherein the circuitry is further configured to issue a search query to the database using the digital identification code as a key to acquire product information.

4. The system according to claim 1, wherein the circuitry is further configured to convert the query data into a distributed representation vector using an encoder of the data generation model, and to search for a record having a reference vector closest to the distributed representation vector using an approximate nearest neighbor search algorithm.

5. The system according to claim 1, wherein the urgency score comprises a probability value calculated by a classification model that performs analysis on the query data, and wherein when the urgency score exceeds a predetermined threshold, the circuitry generates the action proposal as a high-priority alert transmitted to the client terminal.

6. The system according to claim 1, wherein the circuitry is further configured to estimate the emotion by inputting at least one of voice data, a face image, text input, or biometric sensor data received from the client terminal into the emotion identification model, and to adjust a recording timing of the structured data based on the estimated emotion, such that when the estimated emotion indicates stress, the circuitry automatically records the structured data, and when the estimated emotion indicates relaxation, the circuitry provides an option for manual recording.

7. The system according to claim 1, wherein the circuitry is further configured to determine a priority of information to be stored in the database based on the estimated emotion, such that when the estimated emotion indicates stress, the circuitry preferentially stores high-importance information and postpones other information, and when the estimated emotion indicates urgency, the circuitry stores only the most important information.

8. The system according to claim 1, wherein the circuitry is further configured to analyze a past history of records stored in the database associated with the user to predict a next record entry and optimize an input interface based on the prediction, the prediction being performed using at least one of a time-series data analysis algorithm or an association analysis algorithm.

9. The system according to claim 1, wherein the circuitry is further configured to receive, from the client terminal via the communication interface, attribute data of the user comprising at least one of a current health condition or a lifestyle habit, and to filter the reference vectors stored in the database based on the attribute data to exclude records having a contraindication with the attribute data.

10. The system according to claim 1, wherein the circuitry is further configured to receive geographic location information of the user from the client terminal via the communication interface, and to preferentially identify records from the database that are associated with a geographic region corresponding to the geographic location information.

11. The system according to claim 1, wherein the circuitry is further configured to receive social media activity data of the user from the client terminal via the communication interface, extract keywords from the social media activity data using a named entity recognition model, and associate the extracted keywords with the structured data stored in the database.

12. The system according to claim 1, wherein the circuitry is further configured to adjust a method of identifying the record based on the estimated emotion, such that when the estimated emotion indicates stress, the circuitry applies a filtering condition that returns a reduced number of results with high relevance, and when the estimated emotion indicates relaxation, the circuitry returns a wider range of results.

13. The system according to claim 1, wherein the circuitry is further configured to refer to a history database comprising past query data and corresponding outcomes associated with the user, and to calculate a fitness score for each candidate record using a recommendation model comprising at least one of collaborative filtering or matrix factorization.

14. The system according to claim 1, wherein the circuitry is further configured to adjust a presentation method of the inference data based on the estimated emotion, such that when the estimated emotion indicates anxiety, the circuitry generates the inference data in a simplified expression style, and when the estimated emotion indicates relaxation, the circuitry generates the inference data in a detailed expression style.

15. The system according to claim 1, wherein the circuitry is further configured to determine a severity level of the query data using a multi-class classification model, and to select, based on the severity level, one of a plurality of response types comprising a normal recommendation, a facility search and guidance, or an emergency action proposal.

16. The system according to claim 1, wherein the circuitry is further configured to refer to a past history of inference data generated for the user and stored in the database, and to select a format of the inference data based on the past history by analyzing content trends and user preferences extracted from the past history.

17. The system according to claim 1, wherein the circuitry is further configured to determine a priority of generating the inference data based on the estimated emotion, such that when the estimated emotion indicates stress, the circuitry preferentially generates inference data having a high importance attribute, and when the estimated emotion indicates relaxation, the circuitry generates all inference data equally.

18. A system comprising:
a communication interface configured to communicate, via a packet-switched network conforming to at least one of a 5G, Wi-Fi, or Bluetooth communication standard, with a client terminal comprising a touch panel, a microphone, a speaker, a camera having a CMOS image sensor, and a display;
a processor;
a random-access memory;
a memory storing a data generation model obtained by deep learning on a neural network, and an emotion identification model;
a database; and
circuitry configured to:
receive, from the client terminal via the communication interface, image data captured by the camera having the CMOS image sensor;
extract structured data from the image data by performing at least one of optical character recognition using a convolutional neural network or code decoding, and store the extracted structured data in the database;
receive, from the client terminal via the communication interface, query data comprising at least one of text data input via the touch panel or voice data captured by the microphone;
convert the query data into a feature vector using an encoder of the data generation model, calculate a similarity between the feature vector and reference vectors stored in the database, and identify a record from the database based on the calculated similarity;
estimate an emotion of the user by applying the emotion identification model to at least one of the voice data captured by the microphone or image data captured by the camera;
generate, using the data generation model, inference data comprising at least one of a recommendation text or an action proposal, based on the identified record and an urgency score calculated from the query data;
adjust at least one of a presentation method or a level of detail of the inference data based on the estimated emotion; and
transmit the inference data to the client terminal via the communication interface and the packet-switched network, the inference data causing the client terminal to present the inference data to the user via at least one of the display or the speaker.

19. The system according to claim 18, wherein the data generation model comprises at least one of a text generation AI, an image generation AI, or a multimodal generation AI, and wherein the data generation model is a fine-tuned model configured to output inference results from prompts without instructions.

20. A method performed by circuitry of a data processing system comprising a processor, a random-access memory, a memory storing a data generation model obtained by deep learning on a neural network and an emotion identification model, a database, and a communication interface, the method comprising:
receiving, from a client terminal via the communication interface and a packet-switched network, image data captured by a camera of the client terminal;
extracting structured data from the image data by performing at least one of optical character recognition or code decoding using a convolutional neural network, and storing the extracted structured data in the database;
receiving, from the client terminal via the communication interface, query data comprising at least one of text data or voice data;
converting the query data into a feature vector, calculating a similarity between the feature vector and reference vectors stored in the database, and identifying a record from the database based on the calculated similarity;
estimating an emotion of the user by applying the emotion identification model to sensor data received from the client terminal via the communication interface;
generating, using the data generation model, inference data comprising at least one of a recommendation text or an action proposal, based on the identified record and an urgency score calculated from the query data; and
transmitting the inference data to the client terminal via the communication interface and the packet-switched network, the inference data causing the client terminal to present the inference data to the user.
